(19) United States
(12) Patent Application Publication
(10) Pub. No.: US 2026/0284239 A1
(43) Pub. Date: Sep. 24, 2026

(54) DRUG DELIVERY SYSTEMS

(71) Applicant: ANTAROS TRACER AB, Mölndal (SE)

(72) Inventors: Olof Eriksson, Uppsala (SE); John Löfblom, Tullinge (SE); Jonas Persson, Stockholm (SE); Olov Korsgren, Uppsala (SE); Stefan Ståhl, Stockholm (SE)

(21) Appl. No.: 19/473,684

(22) PCT Filed: Apr. 10, 2024
(86) PCT No.: PCT/EP2024/059742

§ 371 Date: Oct. 8, 2025

(30) Foreign Application Priority Data
Apr. 11, 2023 (SE) 2350412-9

(51) Int. Cl. A61K 51/08; C07K 14/705

(52) U.S. Cl. CPC A61K 51/088; C07K 14/70503; C07K 2317/92; C07K 2318/20

(57) ABSTRACT

The current disclosure relates to binding molecules targeting DGCR2 are provided, which may be include and agent, a drug or imaging agent. The conjugated binding molecules may be used as drug delivery systems targeting beta-cells, and may be used in treating beta-cell related disorders, such as diabetes. The conjugated binding molecules may be also used in diagnosis or medical imaging.

### TECHNICAL FIELD

The present disclosure relates to binding molecules targeting DGCR2. The binding molecules may be novel binding polypeptides that specifically bind DGCR2, or conjugated binding molecules, where a drug and/or imaging agent may be attached to the N or C terminal of the binding polypeptides. The binding polypeptides, or conjugated binding molecules, may be used as drug delivery systems targeting beta-cells, and may thus be used in treating beta-cell related disorders, such as diabetes. The conjugated binding molecules may be also used in diagnosis or medical imaging.

### BACKGROUND

Curative treatments of beta-cell related disorders, such as Type 1 Diabetes (T1D) remain elusive. Despite several preclinical successes in the development of pharmaceutical agents which protect or even reverse the loss of beta-cell mass in T1D models, such treatments have not been easily transferred to the clinical situation and patient benefit. Thus, enhanced methods for treatment of diabetes and other beta-cell related disorders are needed.

### SUMMARY

An object of the present disclosure is to provide enhanced methods for drug delivery using novel binding molecules, which may be used for targeted drug delivery as well as in diagnosis and medical imaging. This object is obtained by a binding polypeptide that specifically binds DGCR2, wherein the DGCR2-binding polypeptide comprises a DGCR2 binding motif, DBM, which motif consists of the amino acid sequence selected from: i) EQX3HARNEILX11LPNLNRMQX20X21AFIX25 (SEQ ID NO: 1), wherein, independently of each other, X3 is selected from T and Y; X11 is selected from Q and R; X20 is selected from K, R and H; X21 is selected from S, M, L or T; X25 is selected from R, Q, or K;
and ii) an amino acid sequence which has at least 88% identity to the sequence defined in i), such as 92% or 96% identity thereto.

In one aspect is provided a conjugated binding molecule comprising i) at least one binding polypeptide as defined above and below, and ii) at least one agent, wherein the at least one agent is or comprise one or more of a therapeutic agent and a detectable agent.

The at least one therapeutic agent may be an agent used for treatment of diabetes or beta-cell dysfunction, and may be selected from one or more of small molecules, peptides, proteins, biologic drugs, nucleic acids and cells (cell therapy), such as mesenchymal stem cells, extracellular vesicles and regulatory T cells.

In one aspect is provided a drug delivery system for selectively targeting DGCR2 expressing cells, such as beta-cells, of a subject in need thereof, the drug delivery system comprising a binding polypeptide, or a conjugated binding molecule, as defined above and below, wherein the binding polypeptide is conjugated to at least one therapeutic agent.

In a further aspect is provided a cell engineered to express a chimeric antigen receptor (CAR), wherein the CAR comprises a binding domain, a transmembrane domain connected to the binding domain by a hinge region, and an intracellular domain optionally connected to one or more co-stimulatory domains, wherein the binding domain comprises the binding polypeptide as described above and below.

In a further aspect is provided a pharmaceutical composition containing a conjugated binding protein, or a cell, as described above and below, and a pharmaceutically acceptable carrier or excipient.

In a further aspect is provided a conjugated binding protein, a cell, or a pharmaceutical composition thereof, as described above and below, for use in therapy. The therapy may be therapies of beta-cell related disorders.

In a further aspect is provided a conjugated binding molecule comprising at least one detectable agent, as described above and below, for use in Position Emission Tomography (PET) imaging, or for use in the assessment of beta-cell status in a subject, and/or for diagnosis of beta-cell relates disorders. The at least one detectable agent may be selected from the group consisting of radioisotopes, contrast agents, enzymes, fluorescent molecules, dyes, digoxigenin, and biotin.

### BRIEF DESCRIPTION OF THE DRAWINGS

The above, as well as additional objects, features and advantages of the present inventive concept, will be better understood through the following illustrative and non-limiting detailed description of different embodiments of the present inventive concept, with reference to the appended drawings, wherein:

FIG. 1 shows a schematic description of the Z-molecule scaffold, where FIG. 1A (left) shows the molecular handles available at the C and N terminals and FIG. 1B (right) shows the variable 13 residues.

FIG. 2 is a schematic overview of the drug delivery system, where Z_{AM106} (the DGCR2 binding polypeptide) acts as a guiding system to deliver a drug candidate by binding to DGCR2 on the surface on the beta cells.

FIG. 3 schematically illustrates conjugated binding molecules of the present disclosure, where FIG. 3A illustrates the basic principle of the conjugated molecule comprising a binding polypeptide and an agent as payload, while 3B illustrates a linker, and the use of several agents bound to one polypeptide.

FIG. 4 shows FACS of the E. coli displayed affinity maturation library from example 1 (4 cycles).

FIG. 5 shows results from circular dichroism spectroscopy. FIG. 5A-D illustrates representative circular dichroism spectra for a subset of Z-molecules (Z_{DGCR2:3D1}, Z_{DGCR2:AM106}, Z_{DGCR2:AM115} and Z_{DGCR2:AM156}) at 20° C. before and after heat induced denaturation. FIG. 5 E-H shows thermal stability evaluation by variable temperature measurement of ellipticity between 2° and 100° C.

FIG. 6 illustrates representative SPR sensorgrams for a subset of Z-molecules (Z_{DGCR2:3D1}, Z_{DGCR2:AM106}, Z_{DGCR2:AM115} and Z_{DGCR2:AM156}) demonstrating binding to surface immobilized DGCR2 at different concentrations (1 nM, 5 nM, 25 nM, 50 nM and 100 nM).

FIG. 7 illustrates binding of PET tracer to recombinant human DGCR2. FIG. 7A shows the binding signal of [⁶⁸Ga]Z_{DGCR2:AM106} (Copper coated plates ELISA-based binding of [⁶⁸Ga]Z_{DGCR2:AM106}) and FIG. 7B [¹⁸F]Z_{DGCR2:AM106} to recombinant human DGCR2 protein (p<0.0001) (Copper coated plates ELISA-based binding of [¹⁸F]Z_{DGCR2:AM106}).

FIG. 8 illustrates in vivo SPECT/CT and biodistribution of Indium-111 labeled peptides in rats. FIG. 8A shows rat SPECT/CT scans for each evaluated Z-molecule. FIG. 8B shows kidney retention of each evaluated Z-molecule over time. FIG. 8C shows endpoint organ biodistribution of each evaluated Z-molecule.

FIG. 9 shows in vivo PET/CT and organ biodistribution of [⁶⁸Ga]Z_{DGCR2:AM106} in rats. FIG. 9A illustrates coronal section of the rat PET/CT scans after administration of [⁶⁸Ga]Z_{DGCR2:AM106}. FIG. 9B shows biodistribution of [⁶⁸Ga]Z_{DGCR2:AM106} in each individual organ over time. FIG. 9C shows endpoint biodistribution of each individual organs after administration of [⁶⁸Ga]Z_{DGCR2:AM106}.

FIG. 10 shows in vivo PET/CT and organ biodistribution of [¹⁸F]Z_{DGCR2:AM106} in rats. FIG. 10A shows PET/MRI coronal images of a rat after administration of [¹⁸F]Z_{DGCR2:AM106} through the tail vein. FIG. 10B shows endpoint organs biodistribution of [¹⁸F]Z_{DGCR2:AM106}.

FIG. 11 shows in vivo PET/CT imaging of [¹⁸F]Z_{DGCR2:AM106} in pigs. FIG. 11A shows PET/MRI coronal images of a pig after venous administration of [¹⁸F]Z_{DGCR2:AM106}. FIG. 11B shows time activity bar graph of the [¹⁸F]Z_{DGCR2:AM106} biodistribution in various abdominal tissues. FIG. 11C shows PET/MRI transversal image of the pig after venous administration of [¹⁸F]Z_{DGCR2:AM106}.

FIG. 12 shows PET images of [¹⁸F]Z_{AM106} binding in mice with intramuscular islet grafts in the neck. The crosshairs show the increased signal at the site of the islet graft. FIG. 12A shows a transaxial projection and FIG. 12B shows a coronal projection.

FIG. 13 shows the distribution with time, where 13A shows the in vivo distribution after 3, 10 and 30 minutes, 13B shows a graph of the distribution in different tissues with time, and where 13C shows an increase in [¹⁸F]Z_{DGCR2:AM106} uptake in the SC-islets vs muscles.

FIG. 14 shows the volume of distribution for [¹⁸F]Z_{DGCR2:AM106}, which is a measurement of uptake corrected for background blood signal, in SC-islets, muscles and spleen.

FIG. 15 shows [⁶⁸Ga]Z_{AM106} binding to SC-islets and negative control CHO cells.

The figures are not necessarily to scale, and generally only show parts that are necessary in order to elucidate the inventive concept, wherein other parts may be omitted or merely suggested.

### DETAILED DESCRIPTION

The present disclosure relates to new binding polypeptides, which selectively and/or specifically bind DGCR2, and conjugated binding molecules thereof, which carry therapeutic and/or imaging agents. The conjugated binding molecules may be used in therapy or in imaging applications, for in vitro and in vivo diagnosis. Thus, an aim of the present disclosure is to provide new and enhanced binding polypeptides and conjugated binding molecules specific for DGCR2, which may be used in therapy, diagnosis and medical imaging of beta-cell related disorders.

A critical challenge in all drug development, and a major cause of failure, is the balance between sufficient drug exposure at the target receptor and the emergence of off-target effects during dose escalation. Additionally, drug target receptors may be present in several tissues, where activation generates differing (and sometimes unwanted) effects in different tissues.

Beta-cells are cells that make insulin, a hormone that controls the level of glucose in the blood. Beta-cells are found in the pancreas within clusters of cells known as islets. In some disorders, the beta-cells are destroyed, or do not function properly, such as in diabetes.

Diabetes (diabetes mellitus) is a group of common endocrine diseases characterized by sustained high blood sugar levels. Diabetes is due to either the pancreas not producing enough insulin, or the cells of the body not responding properly to the insulin produced. Diabetes is a disease that is characterized by high blood glucose for an extended period of time, which can lead to long-lasting damage to blood vessels, kidneys and nerves. The two major forms of the disease, Type 1 (T1D) and Type 2 (T2D) are both characterized by the decrease of insulin-producing beta cells located in the pancreatic islets.

Type 1 diabetes (T1D), is an autoimmune disease that originates when the beta-cells that make insulin are destroyed by the immune system. Type 2 diabetes (T2D), is a form of diabetes mellitus that is characterized by high blood sugar, insulin resistance, and relative lack of insulin. As of today, there is no widely-accepted cure for most cases of diabetes. The most common treatment for type 1 diabetes is insulin replacement therapy (insulin injections). Anti-diabetic medications such as metformin and semaglutide, as well as lifestyle modifications, can be used to prevent or respond to type 2 diabetes.

Several surface markers restricted to the beta-cells have been described, often for applications in cell sorting and molecular imaging of beta-cells. However, these markers are usually only specific for the beta-cell within the pancreas, i.e. they are also present in other tissues. Thus, targeting the same markers for guided drug delivery will likely result in off-target drug delivery which could lead to unacceptable toxicity or dosing limitations.

In the context of diabetes treatment, the new class of Glucagon Like Peptide-1 Receptor (GLP1R) agonists is a good example. The GLP-1 receptor is also present in the circumventricular organs (CVOs) around the brain as demonstrated by centrally GLP1R mediated effects of e.g. semaglutide. GLP1R agonism in the beta-cells in the pancreas produce the incretin effect and increased insulin secretion in response to hyperglycemia. GLP1R agonism in the CVOs, on the other hand, is involved in appetite control and at higher exposures nausea. An important reason for discontinuing taking GLP1R agonists (and even withdrawal from GLP1R drug development trials) is gastrointestinal adverse event and nausea, highlighting the importance of achieving sufficient exposure in the target tissue (pancreas), before activity in other tissues or off-target effects lead to unacceptable side effects.

Similarly, GPR44, which is a promising imaging target for pancreatic beta-cells, is also present in the spleen, bone marrow and certain immune cells. Thus, for drug delivery systems intended for beta-cell targeting, special care must be taken to ensure low expression also in other tissues. The properties of the guiding moiety are also important to consider. Firstly, the affinity to its target must be very high, preferably in the nanomolar range, for sufficient accumulation and retention in the target tissue. Secondly, the size of guiding moiety is of importance, where antibodies may be too large (especially if the drug conjugate is small) and result in slow targeting and clearance. The polypeptides of the present invention, on the other hand, is around 20 times smaller than monoclonal antibodies and are generally considered as having faster tissue penetration and clearance.

Thus, there is an urgent need for facile and flexible drug delivery systems to increase drug exposure in the pancreas, while minimizing the off-target and off-tissue effects and toxicity. Such delivery systems may enable re-assessment of existing and emerging drug targets and candidates, which previously have been associated with and discarded due to unacceptable side effects.

Thus, herein is disclosed a novel drug delivery system, which overcomes several of the challenges associated with beta-cell selectivity and off-target toxicity. Such a delivery system is realized by the binding polypeptides and conjugated binding molecules of the present disclosure, which selective and/or specifically bind DGCR2, which in adult humans has a beta-cell specific expression, making it an ideal target protein for directed drug delivery, while minimizing unintended off-tissue and off-target exposure.

Even though the idea of targeting DGCR2 has been previously disclosed, no realization of this idea has yet been put to practice, and no good binders have been developed. For example, U.S. Pat. No. 20,150,259416 A1 describes hypothetical methods for increasing drug delivery to human beta cells (e.g. of Apolipoprotein III). It is based on the finding that local islet production of Apolipoprotein CIII is a novel factor for mediating beta-cell apoptosis in insulin resistant states of T2DM. Thus, methods for inhibiting Apolipoprotein CIII are proposed, where all example figures relate to experiments showing the impact of Apolipoprotein CIII on diabetes and insulin resistance. A composition, A-B-C, is suggested, where A is a pancreatic beta-cell targeting moiety, B is an inhibitor of Apolipoprotein CIII, protein kinase A, Src kinase, or 31 integrin, and C is an optional compound that permits cell entry. As the intended target, a wide range of proteins expressed by human beta cells are listed, among them DGCR2, but no binding moieties or sequences are presented, nor any experiments relating to such. The sequences presented therein only defines the targets they want to bind to, such as DGCR2, but no actual binding moieties are shown, and no data is shown on the actual application of drug delivery for any of the suggested targets. Thus, the skilled person would not obtain any guidance towards DGCR2-guiding moieties from this disclosure.

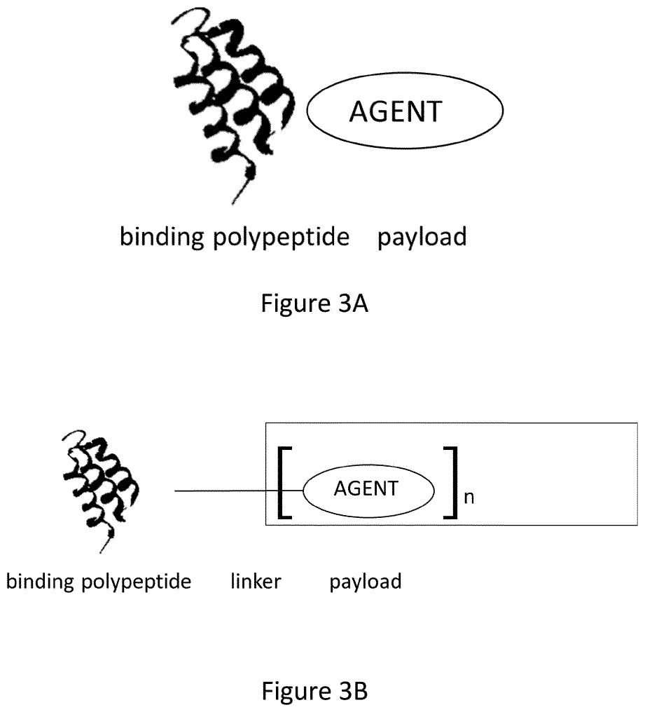

Andersson K G et al (Biotechnol. J. 2019, 14, pages 1-8) discloses affibody molecule generation using autotransporter-mediated display of a Naïve affibody library on the outer membrane of E-coli, which suggest that the method has potential as a complement to phage display. Even though DGCR2 is mentioned in this study as a target of interest amongst others, the identified molecule was a poor binder and the affinity was not sufficient to detect binding at 50 nM concentration of target (FIG. 3 and Figure S3), which is insufficient for further development. Thus, the study does not disclose an enabling binder for DGCR2, which further supports that obtaining such a binder may not be straightforward. Additionally, the peptide sequence for the binder was not identifiable or included in the article. Thus, no explicit affibody binder for DGCR2 is neither disclosed. Accordingly, no enabling binders to DGCR2 are shown in the art.

On the contrary, the present disclosure discloses high-affinity binders towards DGCR2, wherein the current disclosure exhibits a dissociation constant Kp of 30 nM or lower, demonstrating the first binders with sufficient interaction for further use as carriers for drug delivery or imaging reagents to human beta cells.

Aspects of the present disclosure will be described more fully hereinafter with reference to the accompanying drawings. The binding polypeptides, conjugated binding molecules and methods disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

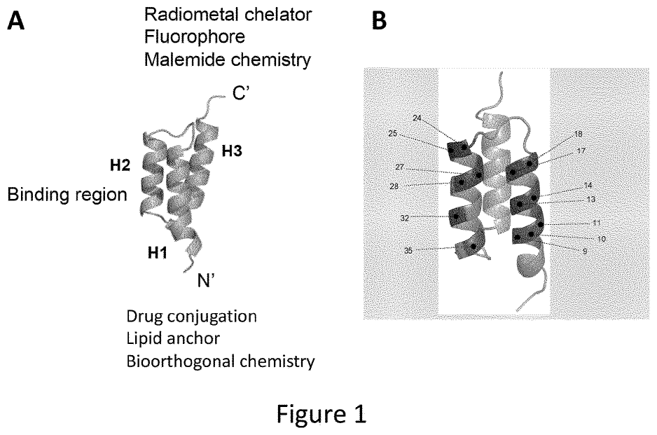

In some embodiments a non-limiting term “binding polypeptide” is used. In some aspects, “binding molecule” may be used interchangeably. The term “binding polypeptide” or “binding molecule” is used herein to denote a polypeptide/molecule that selectively binds to the extracellular domain of DGCR2, i.e. a DGCR2 binding polypeptide. The DGCR2 binding polypeptides of the present disclosure comprise a DGCR2 binding motif, DBM, wherein the identified DGCR2-binding motif, or “DBM”, corresponds to the target binding region of a scaffold, which region constitutes two alpha helices within a three-helical bundle protein domain. A molecule based on this scaffold may be referred to as a Z-molecule. Said scaffold and type of molecules are described, for example, in WO2009/077175, which is incorporated for reference herein to the extent allowed. FIG. 1 shows a schematic description of the Z-molecule scaffold, the molecular handles available at the C and N terminals and the variable 13 residues, which are designed to bind human recombinant DGCR2.

The “conjugated binding molecules” as described herein comprise i) at least one binding polypeptide, and ii) at least one agent, where the at least one agent is joined to the binding protein, such as directly or indirectly joined to the binding protein. The binding polypeptide is conjugated/linked to an agent to form a conjugated binding molecule. The conjugated binding molecule of the invention may be used as an alternative to e.g., conventional antibodies conjugated drugs in various medical, veterinary and diagnostic applications. Thus, the conjugated binding molecule may be used as a therapeutic or diagnostic agent. Diagnostic applications include for example molecular imaging in order to reveal, diagnose and examine the presence of a disease in vivo in the body of a mammalian subject. The conjugated binding molecules may be used in targeting therapeutic or diagnostic agents, both in vivo and in vitro, to cells expressing DGCR2, particularly beta-cells. There is thus provided a combination of a DGCR2-binding polypeptide and a therapeutic agent in the conjugated binding molecule. In some embodiments the therapeutic agent is a radionuclide. Conjugated binding molecules carrying radionuclides as the active agent, where the agent is used for diagnosis or therapy of a disease or condition, may be referred to as radiopharmaceuticals.

In some embodiments, the at least one agent is directly linked to the N- or C-terminal of the binding polypeptide. In some embodiments, the conjugated binding molecule may be referred to as a delivery system, where the binding polypeptide is referred to as a “guiding moiety”, guiding the agent to the beta-cells, where it may assert e.g. its therapeutic effect. In some embodiments, the at least one agent is being indirectly joined to the binding polypeptide, such as via a linker in the form of a chelator. Thus, in one aspect the chelator may be regarded as a linker to carry the radioligand. In other aspects, the linkage between the binding molecule and the chelator/“chelator cage”, may be referred to as a linker. The chelator may be directly or indirectly joined to the binding polypeptide, such as by using a malemide group. In some embodiments the conjugated binding molecule comprises a binding polypeptide to which a chelator is joined via a linker, which linker may be of variable length and composition. Depending on the length, the linker may thus also function as a spacer.

Chelation is a type of bonding of ions and molecules to metal ions. It involves the formation or presence of two or more separate coordinate bonds between a polydentate (multiple bonded) ligand and a single central metal atom. These ligands are called chelants, chelators, chelating agents, or sequestering agents. Chelate complexes of gadolinium are often used as contrast agents in MRI scans, and bifunctional chelate complexes of zirconium, gallium, fluorine, copper, yttrium, bromine, or iodine are often used for conjugation to monoclonal antibodies or peptides for use in antibody or peptide-based PET imaging. These chelate complexes often employ the usage of hexadentate ligands such as desferrioxamine B (DFO), and the gadolinium complexes often employ the usage of octadentate ligands such as DTPA. For radiopharmaceuticals, chelators such as derivatives of 1,4,7,10-tetraazacyclododecane-1,4,7,10, tetraacetic acid (DOTA), derivatives of deferoxamine B (DFO), derivatives of diethylenetriaminepentaacetic avid (DTPA), and derivatives of S-2-(4-Isothiocyanatobenzyl)-1,4,7-triazacyclononane-1,4,7-triacetic acid (NOTA) are commonly used.

Molecular imaging combining imaging agents with targeting moieties in the form of a binding molecule may be used to specifically image diseased beta-cells in the body. The binding polypeptides may be used in molecular imaging to target imaging agents, such as radionuclides, to the cell of interest in vivo. This gives the ability to monitor disease progression and to predict response to a specific therapeutic agent, thus enabling diagnostics and response prediction. The agents may also be referred to as “markers” or “tracers” in this regard. A distinctive feature of both type 1 and type 2 diabetes is the waning of insulin-secreting beta cells in the pancreas. Methods for quantifying the beta-cell mass using e.g. imaging techniques such as Positron Emission Tomography (PET) would thus greatly improve our understanding of diabetes etiology, underlying the importance of finding reliable imaging markers and developing corresponding tracers, as the ones provided herein. Molecular imaging and particularly nuclear medicine have may be used as tools for visualizing and quantifying the beta-cell mass (BCM) in a non-invasive manner. Accurate estimation of the BCM would thus greatly improve our understanding of diabetes etiology as well as the ability to evaluate therapeutic efficacy.

As used herein, the term “capable of binding X”, wherein X is a beta-cell, an extracellular domain of DGCR2 on a beta-cell, refers to a property of a binding polypeptide or molecule of the present disclosure, which may be tested for by use of surface plasmon resonance (SPR) technology. More specifically, the terms “DGCR2-binding” and “binding affinity for DGCR2” as used in this specification refers to a property of a polypeptide which may be tested for example by the use of surface plasmon resonance technology, such as in a Biacore instrument (GE Healthcare). For example, DGCR2-binding affinity may be tested in an experiment in which DGCR2 (e.g., human DGCR2), or a fragment of DGCR2, such as the extracellular domain, is immobilized on a sensor chip of the instrument, and the sample containing the polypeptide to be tested is passed over the chip, wherein the DGCR2 or fragment thereof used may for example comprise an amino acid sequence of DGCR2 extra-cellular domain or SEQ ID NO: DGCR2. If a quantitative measure is desired, for example to determine a K_{D} value for the interaction, surface plasmon resonance methods may also be used. Binding values may for example be defined in a Biacore 2000 instrument (GE Healthcare). The skilled person is aware of said methods and others. It was demonstrated binding polypeptides of the present conjugated binding molecules have certain K_{D} values, that the DGCR2-binding polypeptides bind to DGCR2, such that the K_{D} value of the interaction is at most 1×10⁻⁶ M (1 μM), K_{D}<1 μM. In some examples, the K_{D} value is less than 1 UM but larger than 30 nM (30×10⁻⁹ M), such that 30 nM<K_{D}<1 μM. The targeting polypeptide according to SEQ ID NO: 11 for example has an approximate K_{D} of 2.4 nM, as assessed with SPR.

The skilled person is aware of said method and others. The term “specificity”, sometimes referred to as “selectivity,” of the binding polypeptide for a target refers to a binding polypeptide which will bind to the target with high affinity, but typically not to other molecules. A selective or specific binding polypeptide will not, or to a low extent, cross-react with other targets than the intended target. Thus, by binding “specifically” it is meant that the binding polypeptide binds to its target (i.e. DGCR2) in a manner that can be distinguished from binding to non-target molecules, more particularly that the binding protein binds its target (DGCR2) with greater binding affinity than with which it binds other molecules. That is, the binding polypeptide does not bind to other, non-target, molecules, or does not do so to an appreciable or significant degree, or binds with lower affinity to such other molecules than with which it binds DGCR2. A binding protein “that specifically binds” DGCR2 or “that selectively binds” DGCR2 may alternatively be referred to as “directed against” or “that recognizes” DGCR2.

The binding polypeptides of the present invention may be conjugated to an agent to form a conjugated binding molecule, also referred to as a conjugated binding protein. In one aspect, the agent may be a detectable agent, such as a label of some sort, and the conjugated binding molecule may be used in imaging. In other aspects, the conjugated binding molecule may be formed by linking the binding polypeptide to a therapeutic agent. In said case, the conjugated binding molecule may be used in therapy.

By “therapy” as used herein is meant the treatment of any medical condition. Such treatment may be prophylactic (i.e. preventative), curative (or treatment intended to be curative), or palliative (i.e. treatment designed merely to limit, relieve or improve the symptoms of a condition). Thus, “therapy” or “treating” of a disorder, such diabetes, by means of a conjugated binding polypeptide as used herein, are referring to preventing or ameliorating a certain disorder or medical condition, or to cure it. Prophylactic treatment may include the prevention of a condition, or a delay in the development or onset of a condition. An amount adequate to accomplish this is defined as a “therapeutically effective amount”. Effective amounts for a given purpose will depend on the disease or condition to be treated, its severity and the size/weight and general state of the subject. Thus, the conjugated binding molecules as described herein may be used in the treatment/therapy of any condition in which the target antigen is expressed/overexpressed in a subject, to ameliorate said conditions, and may be administered systemically or locally, and by any suitable method known in the art. A subject, as defined herein, refers to any mammal, e.g. a farm animal such as a cow, horse, sheep, pig or goat, a pet animal such as a rabbit, cat or dog, or a primate such as a monkey, chimpanzee, gorilla or human. Most preferably the subject is a human being.

As the present binding molecules are specific for beta-cells, they may deliver therapeutic agents to the beta-cells. Accordingly, the conjugated binding molecules or delivery systems herein may be used to treat “beta related disorders” or “beta related indications” (used interchangeably herein), which refers to any disorder/indication where beta-cell dysfunction and/or beta-cell death occurs, where beta-cells are destroyed, or do not function properly, and give rise to a disorder or disease state. Such disorders include, but are not limited to, diabetes, including both T1D and T2D and monogenic forms of diabetes, cancers, such as insulinoma, and congenital hyperinsulinism (overactive beta-cells). A beta-cell dysfunction may also be treated, where the beta-cell dysfunction has not yet escalated into a disorder, which may be seen both as a therapeutic and prophylactic treatment.

By the term “diagnosis” or “diagnosing” as used herein is meant a process of determining if a disease or condition, is present in a subject tested. A diagnosis, in the sense of diagnostic procedure, can be regarded as an attempt at classification of an individual's condition into separate and distinct categories that allow medical decisions about treatment and prognosis to be made. Subsequently, a diagnostic opinion is often described in terms of a disease or other condition. The initial task is to detect a medical indication to perform a diagnostic procedure, such as detection of any deviation from what is known to be normal. A diagnostic procedure may be performed in vitro, using the binding polypeptides or conjugated binding molecules herein, or it may be performed in vivo, where the binding polypeptides may carry a detectable agent, such as a label, e.g. for imaging. The binding polypeptide may for example be combined with radioactive isotopes for imaging, such as immunoscintigraphy. In some embodiments, “diagnosis” may not be for detecting a distinct disorder, but for detection or assessment of beta-cells or beta-cell number in both healthy and non-healthy subjects. Thus, the conjugated binding molecules may be used for quantifying beta-cell mass using imaging, such as PET.

The conjugated binding molecules may thus be used in medical imaging, which may be used in diagnosis or prognosis. With the term “prognosis” or “prognosing” as used herein is meant a prediction or estimate of the chance of recovery or survival from a disease when treated. The diagnosis/prognosis may also be used to differentiate patient into different subgroups depending on nature of a disorder. It may further be used for treatment planning, i.e. in determining a dosage regimen. In case the conjugated binding protein comprises a radionuclide, dosimetry may be used, where the diagnosis/prognosis could include determining the radiation dose by measurement, calculation, or a combination of measurement and calculation of the absorbed dose (radiation energy deposited in tissue divided by mass of the tissue) via binding/uptake/internalization of the conjugated binding protein. Prognosis include for example detection of beta-cells, such as number and distribution, in a subject for a prognosis of a beta-cell related disorder.

The DiGeorge Syndrome Critical Region Gene 2 (DGCR2) protein is encoded by the DGCR2 gene, and has UniProt numbers P98153 (human) and P98154 (murine). DGCR2 is a surface protein which is strongly expressed in pancreatic beta-cells, while being absent in the exocrine pancreas. DGCR2 is only present in the beta-cells in the islets of Langerhans, and not in e.g. the alpha cells, and that DGCR2 has been shown to disappear in insulin deficient islets in T1D, further supporting a beta-cell restricted expression. Specific expression of DGCR2 within beta cells has previously been reported based on strong immunoreactivity accented around the plasma membrane in islets of non-diabetic individuals, with no visible staining of insulin-negative islets from T1D patients. Further, it has been shown that beta-cell derived Extracellular Vesicles (EVs) can be identified by their selective expression of DGCR2 at the surface. DGCR2 is located at the cell membrane, with a large extracellular region, and thus accessible for binding by proteins. DGCR2 is therefore a promising target for directed delivery of e.g. drug conjugates and imaging reagents in the pancreas. Finally, DGCR2 is not present in any other tissues in human adults, making it an ideal target protein for directed drug delivery, while minimizing unintended off-tissue and off-target exposure.

The exact function of DGCR2 is unknown, but deletion of the gene cause 22q11.2DS (previously known as DiGeorge Syndrome). 22q11.2DS was recently shown to be associated with both defects in the immune system, obesity and Type 2 Diabetes. Furthermore, DGCR2 is localized in a region on chromosome 22, which is the home of several long non-coding RNA sequences that have been identified in pancreatic beta-cells, as well as islet enhancer clusters. DCGR2 has a major extracellular region, as well as a short intracellular signaling region. Importantly, DGCR2 transcription levels in human islets of Langerhans are unchanged by stressors (exposure to for example cytokines or hyperglycemia), indicating it will remain targetable in surviving beta-cells also in individuals with diabetes. Even though DGCR2 has been identified as a beta-cell specific biomarker, lack of high affinity binders remains and issue.

Accordingly, the present disclosure provides a novel drug delivery system comprising binding polypeptides specific for DGCR2, which overcomes several of the challenges associated with beta-cell selectivity and off-target toxicity, as the surface membrane protein DGCR2 is highly beta-cell restricted in the pancreas, while being virtually absent in other human tissues, and the companion guiding moiety of the delivery system, a binding polypeptide as disclosed herein, has excellent affinity and specificity for human DGCR2.

The binding polypeptides herein, used as guiding moieties in a delivery system where the binding polypeptide is joined to an agent, may be used for efficient delivery of agents to beta-cells. The binding polypeptides, a first-in-class library of novel high affinity peptide binders for the extracellular region of DGCR2, are based on a platform of small, versatile, triple helix peptides of 58 amino acids (scaffold proteins). These molecules belong to a class of small peptides based on a scaffold with excellent properties for molecular imaging and drug delivery. Their small size and generally high affinity and specificity, where their small sizes result in fast blood clearance through renal excretion, yield a favorable pharmacokinetic profile and excellent contrast for molecular imaging. They extravasate rapidly from blood and efficiently penetrate tissues, which is an advantageous feature for disease-targeting. Thus, they combine the affinity and selectivity of antibodies, with the beneficial and rapid biodistribution of smaller peptides, making them ideal for in vivo applications including as imaging vectors or guiding moieties, e.g. for drug delivery.

The binding polypeptides are based on a platform of scaffold molecules, often referred to as Z-molecules, have two molecular handles, and can thus be modified at both its N and C terminals without impairing the binding region. Thus, they can e.g. be simultaneously conjugated to a drug compound or a cell membrane at the N-terminus via a suitable linker, as well as with e.g. a reporter or an imaging reagent (a fluorophore or radioactive marker) at the C-terminus, or vice versa. The scaffold molecule consists of three alpha-helixes, where helix 1 and 2 are engineered to provide the binding surface by encompassing 13 variable amino acid residues, which form the binding region, while the rest of the sequence as well as helix 3 are constant. This gives the scaffold both excellent stability and refolding characteristics, as well as flexible binding properties. Importantly, the C-terminal just after helix 3 is located away from the binding surface and thus can be modified for e.g. addition of imaging reagents or drug conjugates without normally perturbing the molecular interactions at helixes 1 and 2. For the present binding polypeptides, these 13 residues are designed to bind human recombinant DGCR2.

The molecules based on this scaffold furthermore have the capacity to internalize following surface receptor binding, and therefore drug delivery systems using this scaffold may not only be used to increase the exposure of a drug conjugate to the beta-cells, but also to deliver the drug into the beta-cell cytosol. A further advantage is that there is extensive evidence of the safety and efficacy of said molecule scaffold in the clinical setting, both as treatment and for tissue specific delivery of imaging reagents to tissue.

Facile and flexible systems for targeted drug delivery to the pancreatic beta-cells would enable conceptually new anti-diabetic therapeutics and re-assessment of earlier drug candidate failures due to off-target toxicity. A solution for achieving tissue specific delivery is to modify the drug with a guiding moiety, which in turn binds to a target exclusively expressed on the cells of interest. There are two critical parts of a successful delivery system, firstly the target protein and secondly the guiding moiety. The target protein must be restricted to the beta-cell not only in the pancreas but also the rest of the body tissues to avoid off-target drug exposure. Then, a guiding moiety must be developed, with high affinity as well as specificity for the target protein.

Furthermore, the guiding moiety must have certain properties, including suitable size, stability, molecular handles for flexible functionalization as well as low off-target interactions and rapid blood clearance. The conjugated molecules of the invention, the proposed Z-molecule-DGCR2 drug delivery system, clearly fulfils all the afore-mentioned criteria and offers a unique opportunity given the beta-cell specificity of the target combined with a unique, small and versatile high-affinity polypeptide as guiding moiety.

Thus, the present disclosure provides binding polypeptides that specifically binds DGCR2, wherein the DGCR2-binding polypeptide comprises a DGCR2 binding motif, DBM, which motif consists of the amino acid sequence selected from:

(SEQ ID NO: 1)
i) EQX₃HARNEILX₁₁LPNLNRMQX₂₀X₂₁AFIX₂₅

wherein, independently of each other,
   X₃ is selected from T and Y;
   X₁₁ is selected from Q and R;
   X₂₀ is selected from K, R and H;
   X₂₁ is selected from S, M, L or T;
   X₂₅ is selected from R, Q, or K;
and
   ii) an amino acid sequence which has at least 88% identity to the sequence defined in i), such as 92% or 96% identity thereto.

In an embodiment, the DMB is selected from sequences as defined by SEQ ID NO: 4-10, as defined in Table 1 below. In a preferred embodiment, the DMB is

(SEQ ID NO: 4)
EQTHARNEILQLPNLNRMQKSAFIR

TABLE 1

| Sequence ID | Sequence | Definition |
|---|---|---|
| SEQ ID NO: 4 | EQTHARNEILQLPNLNRMQKSAFIR | Binding motif-ZAM106 |
| SEQ ID NO: 5 | EQYHARNEILQLPNLNRMQKMAFIQ | Binding motif-ZAM1 |
| SEQ ID NO: 6 | EQTHARNEILQLPNLNRMQKSAFIK | Binding motif-ZAM115 |
| SEQ ID NO: 7 | EQTHARNEILRLPNLNRMOHLAFIQ | Binding motif-ZAM146 |
| SEQ ID NO: 8 | EQTHARNEILQLPNLNRMQRSAFIQ | Binding motif-ZAM149 |
| SEQ ID NO: 9 | EQYHARNEILQLPNLNRMQRSAFIQ | Binding motif-ZAM156 |
| SEQ ID NO: 10 | EQTHARNEILQLPNLNRMORTAFIK | Binding motif-ZAM158 |

In some embodiments, the DGCR2-binding polypeptide comprising the DBM above, binds to DGCR2 such that the K_{D} value of the interaction is at most 1 μM.

The above definition of a class of sequence related, DGCR2-binding polypeptides is based on an analysis of a number of random polypeptide variants of a parent scaffold, which were selected for their interaction with DGCR2 in selection experiments. The DGCR2-binding motif forms part of a three-helix bundle protein domain. Typically, said DGCR2-binding motif essentially forms part of two alpha helices with an interconnecting loop, within said three-helix bundle protein domain. In particular embodiments of the invention, said three-helix bundle protein domain is selected from domains of bacterial receptor proteins. Non-limiting examples of such domains are the five different three-helical domains of Protein A from Staphylococcus aureus, and derivatives thereof. In some embodiments, the DBM is comprised within the protein Z derivative of domain B of staphylococcal protein A. Thus, in some embodiments, the DGCR2 binding motif forms part of a three-helix bundle protein domain, and the DGCR2 binding motif essentially forms part of two alpha helices with an interconnecting loop, within said three-helix bundle protein domain.

In some embodiments, the DGCR2 binding polypeptide comprises an amino acid sequence selected from:

(SEQ ID NO: 2)
VDNKFNK-[DBM]-DPSQSANLLAEAKKLNDAQAPK;
and
(SEQ ID NO: 3)
VDNKFNK-[PBM]-DPSQSANLLAEAKKLNDAQAPKC,

wherein [DBM] is the DGCR2-binding motif as defined above.

In some embodiments, the DGCR2 binding polypeptide comprises an amino acid sequence selected from SEQ ID NO: 11-17, aa defined in Table 2 below.

TABLE 2

| Sequence | | |
|---|---|---|
| ID | Sequence | Definition |
| SEQ ID NO: | VDNKFNKEQTHARNEILQLPNLNRMQKSAFIRSLIDDPSQSANLLAEAKKLNDAQAPK | Sequence |
| 11 | | ZAM106 |
| SEQ ID NO: | VDNKFNKEQYHARNEILQLPNLNRMQKMAFIQSLIDDPSQSANLLAEAKKLNDAQAPK | Sequence |
| 12 | | ZAM1 |
| SEQ ID NO: | VDNKFNKEQTHARNEILQLPNLNRMQKSAFIKSLIDDPSQSANLLAEAKKLNDAQAPK | Sequence |
| 13 | | ZAM115 |
| SEQ ID NO: | VDNKFNKEQTHARNEILRLPNLNRMOHLAFIQSLIDDPSQSANLLAEAKKLNDAQAPK | Sequence |
| 14 | | ZAM146 |
| SEQ ID NO: | VDNKFNKEQTHARNEILQLPNLNRMQRSAFIQSLIDDPSQSANLLAEAKKLNDAQAPK | Sequence |
| 15 | | ZAM149 |
| SEQ ID NO: | VDNKFNKEQYHARNEILQLPNLNRMQRSAFIQSLIDDPSQSANLLAEAKKLNDAQAPK | Sequence |
| 16 | | ZAM156 |
| SEQ ID NO: | VDNKFNKEQTHARNEILQLPNLNRMQRTAFIKSLIDDPSQSANLLAEAKKLNDAQAPK | Sequence |
| 17 | | ZAM158 |

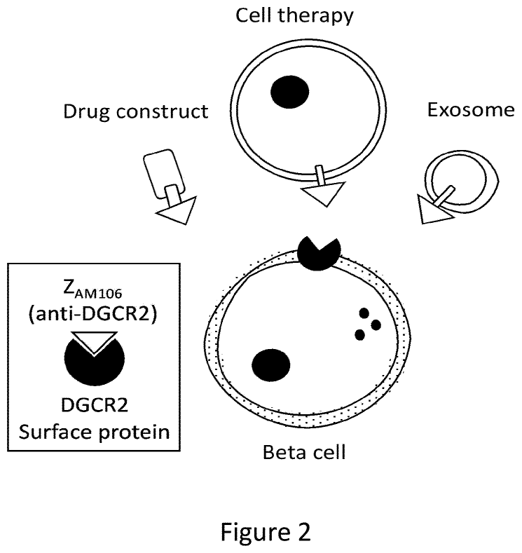

In other embodiments, the DGCR2 binding polypeptide comprises a modified sequence of the binders of SEQ ID NO: 11-17, where a cysteine has been introduced at the C terminus, enabling site-specific labeling using thiol-directed chemistry. These modified polypeptides comprising an amino acid sequence selected from SEQ ID NO: 18-24. In a preferred embodiment, the DGCR2 binding polypeptide is the Z-variant ZAM106, as defined by SEQ ID NO: 11, or a modified variant, as defined by SEQ ID NO: 18. Accordingly, FIG. 2 illustrates is a schematic overview of a drug delivery system, where the binding polypeptide is Z_{AM106}, which acts as a guiding system (guiding moiety of the conjugated binding molecule) to deliver a drug candidate (therapeutic agent) to the pancreas by binding to DGCR2 on the surface on the beta-cells. It should be noted that ZAM106 may also be referred to as ZDGCR2:AM106 herein.

As the skilled person will realize, the function of any polypeptide, such as the DGCR2-binding capacity of the polypeptides according to the invention, is dependent on the tertiary structure of the polypeptide. It is therefore possible to make minor changes to the sequence of amino acids in a polypeptide without affecting the function thereof. Thus, the invention encompasses modified variants of the DBM of i), which are such that the resulting sequence is at least 89% identical to a sequence belonging to the class defined by i), such as at least 88% identical, such as at least 92% or 96% identical. For example, it is possible that an amino acid residue belonging to a certain functional grouping of amino acid residues (e.g., hydrophobic, hydrophilic, polar etc.) could be exchanged for another amino acid residue from the same functional group. Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32 (5): 1792-1797, 2004) may be used for multiple sequence alignments, though any other appropriate programme may be used. Whether the alignment is pairwise or multiple, it must be performed globally (i.e. across the entirety of the reference sequence) rather than locally. Sequence alignments and % identity calculations may be determined using for instance standard Clustal Omega parameters:matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

The skilled person will appreciate that various modifications and/or additions can be made to a binding polypeptide according to the present disclosure in order to tailor the polypeptide to a specific application. For example, a DGCR2 binding polypeptide may be extended by C terminal and/or N terminal amino acids. Said extended polypeptide is a polypeptide which has additional amino acids residues at the very first and/or the very last position in the polypeptide chain, i.e. at the N- and/or C-terminus. The polypeptide may be extended by any suitable number of additional amino acid residues, for example at least one amino acid residue. Each additional amino acid residue may individually or collectively be added in order to, for example, improve production, purification, stabilization in vivo or in vitro, coupling, or detection of the polypeptide. Such additional amino acid residues may comprise one or more amino acid residues added for the purpose of chemical coupling. One example of this is the addition of a cysteine residue. Such additional amino acid residues may also provide a “tag” for purification or detection of the polypeptide such as a His6 tag or a “myc” (c-myc) tag or a “FLAG” tag for interaction with antibodies specific to the tag.

The binding polypeptides described above are advantageous in that they bind well to DGCR2, and hence are beta-cell specific. The polypeptides may in particular bind to the extra-cellular domain of DGCR2. Typically, the polypeptides can be relatively short. By virtue of their small size, they are expected to exhibit a more efficient penetration in tumor and normal tissue than antibodies, as well as to have better systemic circulation properties than monoclonal antibodies (which often have too long circulation times). Thus, they are considered suitable candidates for the development of guiding moieties for therapeutic agents and molecular imaging agents, as in the present disclosure.

Binding polypeptides may be joined/linked to one or more agents as payload as illustrated in FIG. 3 to form conjugated binding molecules, and by recognizing and binding the target, DGCR2, they may be used to treat beta-cell related disorders or assess beta-cell numbers in a subject. It may also be possible to detect and quantify disease states, study distribution of drug/drug candidates in the body, and enable the measurement of efficacy of treatments. In some aspects, the agent (payload) is a detectable or imaging agent, such as a label, and the conjugated binding molecule is used in imaging. The agent may be a radionuclide. The term “radionuclide” may also be referred to as “radioisotope”, and refers to a nuclide that has excess nuclear energy, making it unstable, and prone to undergo radioactive decay. FIG. 3A illustrates the basic principle of the conjugated molecule comprising a binding polypeptide and an agent, while FIG. 3B illustrates a linker, and the use of several agents.

Further disclosed herein are conjugated binding molecules, where the binding protein herein may be joined (directly or indirectly joined), e.g. by genetic fusion, conjugated or chemically linked to an “agent”, i.e. a moiety with a certain property, to form a conjugated or fused binding molecule or binding protein, where the binding polypeptide and agent may be directly joined to one another, such as when coupling an radioisotope to the binding protein, or may be joined via a linker (i.e. joined indirectly to one another), to the binding protein. One or more agents may be jointed/linked to the binding polypeptide, such as being conjugated to one or both of the N-terminus or C-terminus of the binding polypeptide. In some embodiments, the binding molecule comprise at least one therapeutic agent and at least one detectable agent binding, wherein the therapeutic agent is conjugated to one of the N-terminus or C-terminus of the binding polypeptide, and the detectable agent is conjugated to the other of the N-terminus or C-terminus of the binding polypeptide. Thus, the therapeutic agent may be conjugated to the N-terminus to the binding polypeptide and the detectable agent may be conjugated to the C-terminus of the binding polypeptide, or the other way around.

The agent may also be coupled using a chelator (a form of indirect joining), where the chelator is joined to the binding protein and chelates the agent. In one embodiment, the at least one agent is indirectly joined to the binding polypeptide via a linker in the form of a chelator, functional group or prosthetic group, wherein the at least one agent comprises or consists of a radionuclide. In some embodiments the binding molecule may include linkers, such as Tetrazine or Malemide that are not chelators, referred to as a “functional group” or “chemical moiety” herein. A TCO (trans-cyclooctene) functional group may be used, an example being trans-cyclooctene-tetrazine (TCO-Tz) ligation chemistry may be used. Use of “prosthetic groups” includes other common techniques of labelling, such as activated esters, tetrazines, tosylates.

The chelator may be for example selected from the group consisting of derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA), derivatives of (tBu) 4 (1-(1-carboxy-3-carbotertbutoxypropyl)-4,7,10-(carbotertbut-oxymethyl)-1,4,7,10-tetraazacyclododecane) (DOTAGA), derivatives of 1,4,8,11-tetraazacyclodocedan-1,4,8,11-tetraacetic acid (TETA), derivatives of 1,4,7-triazacyclononane, 1-glutaric acid-4,7-acetic acid (NODAGA), derivatives of 1,4,7-Triazacyclononane-1,4,7-triacetic acid (NOTA), and Restrained Complexing Agent (RESCA).

The agent may be a therapeutic agent or a detectable imaging agent. The therapeutic agent may be any agent capable of treating or ameliorating a beta-cell related disorder, including treating beta-cell dysfunction (treat dysfunctional beta-cells). The therapeutic agents or active pharmaceutical ingredients (API) joined, conjugated or linked to the binding protein may be selected from e.g. small molecules, peptides, proteins, biologic drugs, nucleic acids and cells (cell therapies), such as mesenchymal stem cells, extracellular vesicles and regulatory T cells. In some embodiments, the agent is an antidiabetic agent. One or more therapeutic agents may be linked/joined/conjugated to the binding molecule.

The binding molecule may alternatively, or in addition, comprise at least one detectable agent or imaging agent. Molecular imaging combining imaging agents with targeting moieties in the form of a binding molecule may be used to specifically image diseased sites in the body. The binding polypeptides may be used in molecular imaging to target imaging agents, such as radionuclides, to the cell of interest in vivo. This gives the ability to monitor disease progression and to predict response to a specific therapeutic agent, thus enabling diagnostics and response prediction for any tissue and disease where the target is expressed/overexpressed. The imaging/detectable agents may be selected from the group consisting of radioisotopes, contrast agents, enzymes, fluorescent molecules, dyes, digoxigenin, and biotin. The term “radioisotope” may also be referred to as “radionuclide”, and refers to a nuclide that has excess nuclear energy, making it unstable, and prone to undergo radioactive decay. The one or more radioisotopes are each independently selected from the group consisting of ⁶⁸Ga, ¹¹C, ¹⁸F, ⁶⁴Cu, ⁸⁹Zr, ⁴⁴Sc, ⁵²Mn, ⁵⁵Co, ⁷²As, ¹¹¹In, ^{99m}Tc, ⁶⁷Ga, ⁷²As, ¹²³I, ¹²⁴I, ¹²⁵I, ¹²⁴I, ⁴⁷Sc, ¹⁸⁸Re, ²⁰¹Tl, ¹⁷⁷Lu, ⁹⁰Y, Gd, ²¹³Bi, ²²⁵Ac, ²¹²Pb and ²¹¹At. The detectable agent may be detectable by any suitable imaging technique, such as PET, MRI, SPECT, Computed Tomography (CT), optical or ultrasound imaging.

In some aspects is provided a drug delivery system, wherein the binding polypeptide is used as a guiding moiety/delivery agent for delivering therapeutic agents to cells expressing DGCR2. Thus, in one aspect is provided a drug delivery system for selectively targeting DGCR2 expressing cells, such as beta-cells, of a subject in need thereof, the drug delivery system comprising a binding polypeptide of the present disclosure, or a conjugated binding molecule carrying one or more additional agents, wherein the binding polypeptide is conjugated to at least one therapeutic agent. Hence, in some embodiments the drug delivery system comprises, besides from the guiding binding polypeptide and the therapeutic agent, one or more additional agents, such as an additional therapeutic or prophylactic agent, or a detectable agent, such as for imaging of the targeted cells. The system is capable of delivering the therapeutic agent to one or more of the exterior, (islet) microenvironment and cytosol of a DGCR2 expressing cell, such as a beta-cell. Thus, the conjugated binding molecule may be able to both bind to the targeted cell, and internalize into the cell, to provide a therapeutic action. As they have the capacity to internalize following surface receptor binding, the delivery system may further be used to facilitate intracellular transport-which is a requisite for e.g. targeted delivery of RNA and gene therapies, including future applications of emerging CRISPR-CAS9 technologies, in the search for curative treatments of e.g. T1D.

The binding polypeptide of the present disclosure have been engineered to optimize their use as a guiding moiety and for drug delivery, which includes redesigning the scaffold molecular handles at both the C and N terminal, without impairing the DGCR2 binding region or the drug activity. The polypeptides bind to the extracellular portion of DGCR2 and is thus available for targeting and delivery of larger drug conjugates that cannot easily pass the cell membrane. In its miniaturized form, they may be functionalized with an imaging reagent (68Ga or 18F at the C-terminal via DOTA chelation) which has rapid biodistribution and tissue penetration in rats, combined with low off-target binding. In a preferred embodiment, the binding polypeptide is a polypeptide as defined by SEQ ID NO: 11 (also referred to as Z_{AM106}), which has a combination of high affinity, optimal biodistribution (low background in most tissues) and sufficient heat stability/refolding properties. Other polypeptides as used herein may also be used, which have comparable affinity towards DGCR2, but may have a different or overlapping binding site. A variant is to increase affinity by constructing homo-dimers, by coupling two of the DGCR2 targeting polypeptides in sequence, by a suitable linker. This technique has been shown to increase the affinity of other similar molecules by up to a factor of 50, but with the drawback of doubling the molecular size (which may negatively affect biodistribution and clearance).

Modifications (functionalization) to the binding polypeptides (to e.g. create conjugated binding molecules) could be to include imaging reporters (radiometal chelators such as DOTA (⁶⁸Ga, short 68 minutes radioactive half-life)) and DFO (⁸⁹Zr, intermediate, 4 days radioactive half-life) or fluorophores (e.g. Alexa Fluor 647)), via malemide chemistry at the C-terminal cysteine, as well as functional moieties for covalent drug conjugations at the N-terminal. Functionalization may include for example tetrazine or trans-cyclooctene (TCO) groups for biorthogonal chemistry at mild conditions. Linker selection and optimization may be crucial to preserve binding capacity of both the binding polypeptide and the drug agent/conjugate, as the drug agents may include both small molecules and extracellular vesicles (EVs). In other embodiments, the N-terminal of binding polypeptide is modified in order to enable addition of the guiding moiety to extracellular vesicles (EVs), or more generally all cell membranes. It has been demonstrated that autologous transplantation of Mesenchymal Stromal Cells (MSCs) to individuals with T1D preserved, and even improved beta-cell function, and that MSCs probably exert many of their beneficial effects through release of EVs. EVs are thus highly interesting as an emerging therapeutic strategy in T1D, but exposure of the pancreas target tissue will be limited as the EVs are administered systemically. Thus, it is a further object to use MSC derived EVs as a model cell product for modification with a surface tethered binding polypeptide, to improve accumulation in human islets of Langerhans by the addition of the guiding moiety. Thus, enabling guided EVs directed towards human beta-cells.

The binding polypeptides have been shown to selectively bind DGCR2 with a high affinity, and may thus be used as a conjugated binding molecule, for imaging and in drug delivery systems, as described above. The binding polypeptides of the present disclosure may also be used for designing chimeric antigen receptors (CARs) against DGCR2 to obtain DGCR2-targeted CAR cells, such as CAR-T-cells or CAR Regulatory T cell (CAR T-reg cell). For example, a CAR T-cell is made by isolating T cells from a subject, inserting a gene for the CAR in the T-cells to create a CAR T-cell expressing a CAR protein, where the CARs are hybrids of T-cell and receptors comprising 4 distinct regions; an extracellular domain which recognizes the antigen (binding part of binding polypeptide) connected to a transmembrane domain by a hinge (spacer), where the transmembrane domain has a hydrophobic alpha-helix structure, and wherein the transmembrane domain is connected to an endodomain (intracellular domain). The endodomain may also comprise one or more co-stimulatory domains.

Thus, in one aspect is provided a cell engineered to express a chimeric antigen receptor (CAR), wherein the CAR comprises a binding domain, a transmembrane domain connected to the binding domain by a hinge region, and an intracellular domain optionally connected to one or more co-stimulatory domains, wherein the binding domain comprises a binding polypeptide of the present disclosure. The cell may be a human cell, an immune effector cell, such as a T-cell, a T-reg cell, an NK cell or a macrophage. The CAR-T cells may thus be used to treat cancer in the beta-cells, such as insulinoma. Alternatively, CAR T-reg cells may be targeted towards the beta cells by the binding polypeptide of the present disclosure, thereby allowing modulation or suppression of the immunological response in the beta cell microenvironment.

In some aspects is provided a pharmaceutical composition containing a conjugated binding molecule, or a CAR Tcell, and a pharmaceutically acceptable carrier or excipient. As used herein, “pharmaceutically acceptable carrier or excipient” includes any and all solvents, dispersion media, coatings, antibacterial and antifungal agents, isotonic and absorption delaying agents and the like that are physiologically compatible. Preferably, the carrier or excipient is suitable for parenteral, e.g. intradermal, intravenous, intramuscular or subcutaneous administration (e.g. by injection or infusion). Depending on the route of administration, the binding polypeptide or conjugated binding molecule may be coated in a material to protect them from the action of acids and other natural conditions that may inactivate or denature it. Preferred pharmaceutically-acceptable carriers comprise aqueous carriers or diluents. Examples of suitable aqueous carriers that may be employed in the pharmaceutical compositions, kits and products include water, buffered water and saline. Examples of other carriers include ethanol, polyols (such as glycerol, propylene glycol, polyethylene glycol, and the like), and suitable mixtures thereof, vegetable oils and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, sodium chloride and the like.

The conjugated binding molecule, a CAR-cell, or a pharmaceutical composition thereof, may be used in therapy, such as in therapies of beta-cell related disorders. Thus, they may be used in any therapy targeting DGCR2 expressing cells, particularly beta-cells. As an example, they may be used in diabetes therapy, or in insulinoma therapies. The conjugated binding molecules herein may also be used in imaging, such as Position Emission Tomography (PET) imaging. In an example, the conjugated binding molecules may be used in the assessment of beta-cell status in a subject, or diagnosis of beta-cell relates disorders. Thus, they may be used in assessment of diabetes mellitus, including diagnosis, and assessment of stage and status of the disorder. Assessment may also be for understanding diabetes etiology for future therapeutic developments and evaluation of treatment efficacy. The conjugated binding molecules may also be used in detection or assessment of beta-cells, including beta-cell number in health and disease, i.e. healthy and non-healthy subjects.

Accordingly is provided a conjugated binding protein, a cell, or a pharmaceutical composition thereof, as described above and below, for use in therapy. The therapy may be therapies of beta-cell related disorders. Provided herein are methods of treating a subject in need thereof, comprising administering a therapeutically effective amount of a conjugated binding molecule, cell, or pharmaceutical composition to the subject. Thus, a method of treating a subject in need thereof may comprise: administering therapeutically effective amount of a conjugated binding molecule comprising a therapeutic agent (drug compound), wherein the therapeutically effective amount relates to the drug compound, thereby delivering a therapeutic agent to the DGCR2 expressing cells (surface and/or cytosol) bound by the polypeptide of the conjugated binding molecule, or their microenvironment, where in the subject may suffer from a beta cell related disorder, which may include beta cell dysfunction and/or beta cell death.

In a further aspect is provided use of a binding polypeptide as disclosed herein as a guiding moiety for delivering one or more drug compound to DGCR2 expressing cells (surface and/or cytosol) or their microenvironment, i.e. surrounding tissue. In an embodiment is provided use of a conjugated binding molecule, a cell or pharmaceutical composition as disclosed herein, for the treatment, prevention or diagnosis of beta related disorder and beta cell dysfunction.

In a further aspect is provided an in vitro method for detecting expression of DGCR2, the method comprising: (i) contacting a binding polypeptide or a conjugated binding molecule as described herein, to a biological sample, such as a tissue sample or liquid, obtained from a subject, such that the binding polypeptide or conjugated binding molecule binds to DGCR2 if present in the biological sample; (ii) washing the biological sample to remove unbound binding polypeptides or conjugated binding molecules; and (iii) detecting any binding polypeptide or conjugated binding molecule that have bound DGCR2 in the biological sample.

In a further aspect is provided an in vivo method for detecting expression of DGCR2, the method comprising: (i) administering a conjugated binding molecule as described herein, to a subject, wherein the conjugated binding molecule binds DGCR2; and (ii) detecting that the conjugated binding molecule has bound a cell expressing DGCR2.

The conjugated binding molecules, or pharmaceutical compositions comprising them, may be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection. Alternatively, a non-parenteral route may be used, such as a topical, epidermal or mucosal route of administration. Local administration is preferred, including peritumoral, juxtatumoral, intratumoral, intralesional, perilesional, intra cavity infusion, intravesicle administration, and inhalation. However, the conjugate or engineered cells may also be administered systemically.

A suitable dosage of a specific binding molecule or pharmaceutical composition of the invention may be determined by a skilled medical practitioner. Actual dosage levels of the active ingredients in the pharmaceutical compositions and products of the present invention may be varied so as to obtain an amount of the active ingredient which is effective to achieve the desired therapeutic response for a particular subject, i.e. patient, without being toxic to the patient. The selected dosage level will depend upon a variety of pharmacokinetic factors including the activity of the particular conjugate employed, the route of administration, the time of administration, the rate of excretion of the protein, the duration of the treatment, other drugs, compounds and/or materials used in combination with the particular compositions employed, the age, sex, weight, condition, general health and prior medical history of the patient being treated, and like factors well known in the medical arts. Doses may be given at certain intervals, such as bi-weekly (every fortnight). For other purposes, shorter intervals, such as daily doses, may be more suitable. Suitable intervals for different types of treatments would be apparent to the skilled person. The conjugate/composition may be administered in a single dose or in multiple doses. The multiple doses may be administered via the same or different routes and to the same or different locations. Alternatively, they can be administered as a sustained release formulation, in which case less frequent administration is required. Dosage and frequency may vary depending on the half-life of the administered species in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease. In an exemplary dosage regime, the conjugated binding protein is administered to the subject once a week, once a fortnight or once every three weeks, in a cycle repeated from 2 to 10 times. If not curative, the administration may be continuously ongoing at a certain interval.

The present invention provides new enhanced binding polypeptides, which bind human DGCR2. Binding polypeptides may be joined/linked to one or more agents as payload as illustrated in FIG. 3, and by recognizing and binding a target present/overexpressed on target cells, they may either locate the cell if the agent is e.g. an imaging or detectable agent, or they may treat the target cell by carrying a therapeutic agent. The binding molecules/compositions are introduced into the body by various means (such as injection or ingestion) localize the specific antigen (DGCR2), bind the target cell, stay on cell surface or become internalized into the cell, and expose their payload to the tissue. The binding molecules of the present disclosure may be used to treat a number of beta-cell related disorders, which are linked to beta-cell dysfunction or beta-cell death, or potentially other disorders where DGCR2 would be expressed, as discussed above and below. Experimental data, as summarized herein, is further set out in more detail in the example section below.

The content of this disclosure thus enables treatment, imaging and diagnosis of disorders linked to beta-cell dysfunction, such as diabetes, by administering conjugated binding proteins of the invention. In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, products, and systems. It should be appreciated that the example embodiments presented herein may be practiced in any combination with each other. It should also be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be realized in the broadest sense of the claims. All references cited herein are incorporated by reference to the extent allowed.

### EXAMPLES

An object of the present disclosure is to develop novel binding polypeptides, Z-molecules, as guiding moieties to enable beta-cell selective drug delivery, with minimal off-target accumulation, via targeting of DGCR2. Thus, disclosed herein are examples of protein engineering optimization of Z-molecules as guiding moieties, in vitro evaluation of the Z-molecules, in vivo testing of biodistribution in rat and pig and a mouse model of human islet transplantation. Even though all Z-molecules have not been included in all experiments, based on their similarities and determined properties, they are expected to yield similar binding characteristics to DGCR2 in vitro, only with small differences in affinity (i.e. dependent on the concentration), and hence the examples are representative to all the Z-molecules of the present disclosure.

### Example 1, Generation and Selection of the DGCR2 Binding Z-Molecules

### Aims

This example describes the generation and selection of the DGCR2 targeting Z-molecule candidates.

### Methods

Subcloning of the Affinity Maturation Library and Electroporation into E. coli and Selections of Affinity Matured Variants by Fluorescence Activated Cell Sorting (FACS)

To obtain Z-molecules with increased binding potential towards DGCR2, an affinity maturation library was designed by randomizing 13 positions on the binding surface of helix 1 and helix 2. In each randomized position, the DNA library contained a combination of 70% of the original codon belonging to the first-generation DGCR2 binder Z_{DGCR2:3D1} and 30% of a mix of the other codons with the exceptions of cysteine, proline and glycine.

The randomized oligonucleotide sequences encoding helix 1 and 2 of the Z-molecules (Twist Bioscience) were subcloned into the surface display vector. Ligation of the insert into the vector was done by adding a threefold molar excess of insert over vector and CloneSmart DNA ligase. Thirty electroporations were performed in total, divided into groups of 10. After electroporation, the cells were transferred to 10 ml of LB media and incubated for 1 h at 37° C. The cell suspension was thereafter inoculated into 100 mL LB medium containing chloramphenicol and incubated overnight at 37° C. A sample from the pooled cells was diluted and spread on agar plates containing chloramphenicol to determine the total library size. The following day, the culture was centrifuged at 5000×g at 4° C. for 5 min, weighed and resuspended in 15% glycerol. The E. coli library was thereafter stored at −80° C.

The recombinant E. coli displayed affinity maturation library was induced and cells were washed with PBSP buffer (10 mM PBS, pH 7.4 containing 0.1% Pluronic F127). Cells were then resuspended in PBSP containing biotinylated DGCR2 (Glu22-Ala349) purchased from R&D Systems. The mix was incubated on a rotamixer at room temperature (RT) for 1 h, followed by extended washes with ice cold PBSP before resuspending in 150 nM human serum albumin (HSA)-Alexa 647 conjugate and 0.5 μg/ml streptavidin conjugated with R-phycoerythrin (SAPE) (Invitrogen) or neutravidin conjugated with Oregon Green 488 (NAOG) (Life Technologies), followed by incubation on ice for 30 min. The cells were thereafter washed with ice-cold PBSP and resuspended in ice-cold PBSP for sorting in a MoFlo Astrios EQ flow cytometer (Beckman Coulter). The sort gate was set to sort out the top fraction of cells displaying Z variants (typically 0.1%) showing the highest R-phycoerythrin or Oregon Green 488 to Alexa Fluor 647 fluorescence intensity ratio. Bacteria were sorted into a 1.5 ml tube containing LB medium and chloramphenicol. The sorted cells were incubated for 1 h on rotamixer at 37° C. and thereafter inoculated to 50 ml LB medium with chloramphenicol for overnight cultivation.

### Recombinant Expression of the First Generation and Affinity-Matured DGCR2-Binding Z-Molecules

Affinity maturated Z-molecules were subcloned into an E. coli expression vector based on the pETsystem (GenScript Biotech Corp) under the control of a T7 promoter and a N-terminal hexahistidine tag as well as a unique C-terminal cysteine residue. The recombinant proteins were purified using HisPur Cobalt Resin (#89,966, Thermo Scientific) according to the manufacturer's instructions.

### Circular Dichroism Spectroscopy

Thermal stability and refolding after heat induced denaturation were measured for each variant using circular dichroism spectroscopy. All measurements were performed on a Chirascan instrument (Applied Photophysics Ltd) and three spectra were run for each Z-molecule. First, a wavelength spectrum was run to confirm the presence of a helical structure associated with the Z-molecule scaffold. The ellipticity was measured at 20° C. from 195 nm to 260 nm in five technical replicates. The thermal stability was determined by following the ellipticity at 221 nm during variable temperature measurements (5° C./min from 20° C. to 100° C.). After the heat induced denaturation, the samples were cooled to 20° C. before measuring ellipticity from 195 to 260 nm in five technical replicates to check for refolding.

### Surface Plasmon Resonance Studies

DGCR2 recombinant protein was diluted in 10 mM sodium acetate, pH 4.5 and immobilized on a CM5 chip surface using 1-ethyl-3-(3-dimethylaminopropyl) carbodiimide hydrochloride (EDC)/N-hydroxysuccinimide (NHS) coupling chemistry for use as immobilized target in a Biacore 8K instrument (Cytiva). The surfaces were inactivated using ethanolamine prior to binding studies. One surface was activated/inactivated for blank subtraction. A first screening was performed by injecting 100 nM of the respective Z-molecule over the respective immobilized target for 120 s, followed by running buffer for 300 s before regeneration of the surfaces. 10 mM glycine-HCl pH 2.5 were used for regeneration in the experiments. All the Z-molecules selected in the affinity maturation procedure expressed as described above were injected in concentration ranging from 1 nM to 100 nM in triplicates over both surfaces.

### Results

### Affinity Maturation of First Generation DGCR2 Specific Z-Molecules

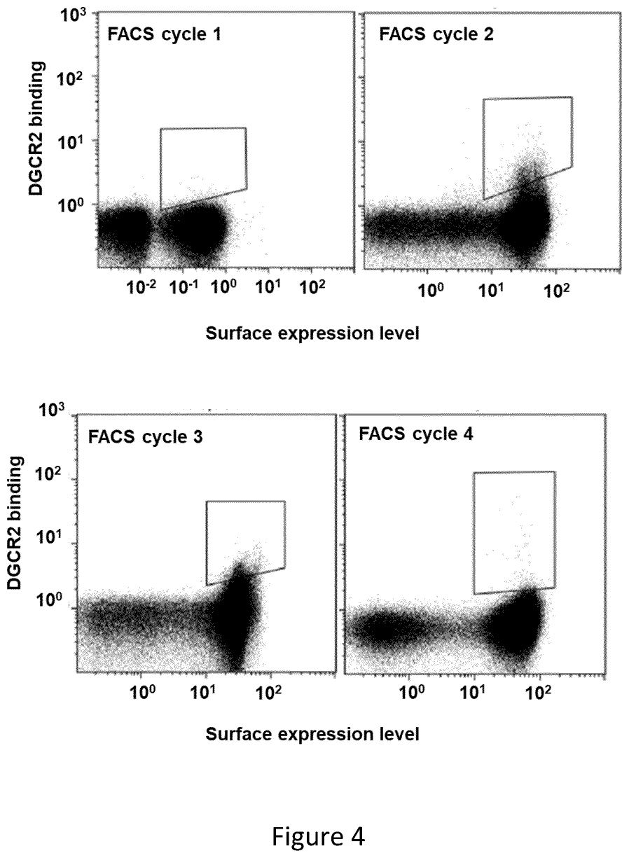

The library was expressed on the surface of E. coli and subjected to four rounds of FACS with alternating rounds of amplification by cell growth. The selection stringency (target concentration, sorting parameters and sorting gates) was increased with each sorting round. The top 0.1% of the library demonstrating the highest ratio of target binding to surface expression was gated and isolated for amplification and subsequent rounds of sorting. The visualization of the target-binding properties of the library in the flow cytometer revealed an enrichment of DGCR2 positive clones in each sorting round (FIG. 4). After the third and fourth selection rounds, cells were spread on semi-solid medium and randomly picked variants were sequenced. Clones appearing more than once among the sequences were selected for further characterization. The results are illustrated in FIG. 4, which shows FACS of the E. coli displayed affinity maturation library. X-axes: fluorescence intensity corresponding to surface expression levels measured by fluorescently labelled HSA. Y-axes: fluorescence intensity corresponding to labelled DGCR2 binding. Sorting gates are indicated in the dot plots.

### Circular Dichroism Spectroscopy

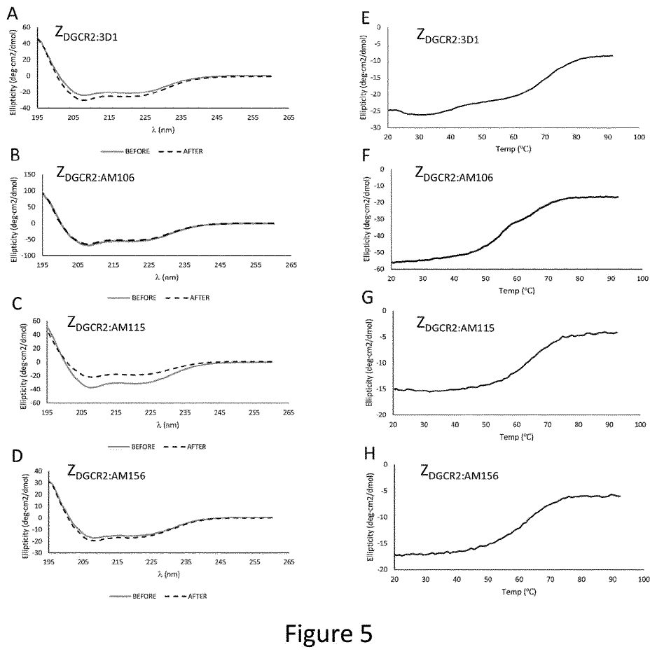

The melting temperature (Tm) for the Z-molecules was determined by circular dichroism (CD) spectroscopy and variable temperature measurements. Measuring the CD spectrum before and after heat induced denaturation demonstrated complete refolding for several of the variants with good thermal stability up to temperatures of around 60° C. for all the evaluated Z-molecules, as shown in FIG. 5 and Table 3 below, Table 3 illustrating melting temperature as determined by CD and equilibrium dissociation constants as determined by SPR.

TABLE 3
Melting T ° and affinity

| Z-molecule | Tm (° C.) | K_{D} (nM) |
|---|---|---|
| ZAM106 | 56.4 | 2.4 |
| ZAM1 | 69.0 | 27.0 |
| ZAM115 | 64.0 | 2.1 |
| ZAM146 | 55.0 | 18.0 |
| ZAM149 | 68.0 | 48.0 |
| ZAM156 | 60.0 | 8.5 |
| ZAM158 | 58.0 | 2.4 |

### Surface Plasmon Resonance Characterization of DGCR2-Binding Z-Molecules

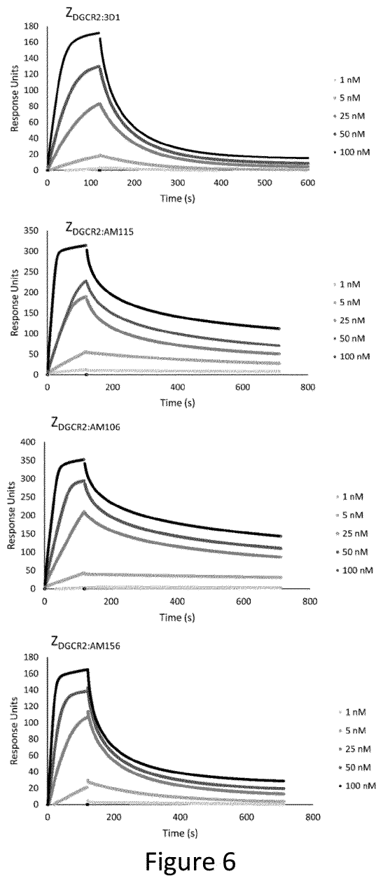

The interactions of all the Z-molecules with DGCR2 were analyzed in surface plasmon resonance (SPR) based biosensor assays and several of the affinity-matured variants were found to have significantly increased affinity towards DGCR2 compared to the primary variant Z_{DGCR2:3D1}, as shown in FIG. 6 and Table 3 above.

### Example 2, Characterization of In Vitro Binding of Radiolabeled Z_{DGCR2:AM106} Z-Molecules

### Aims

This example describes the evaluation of the [⁶⁸Ga]Z_{DGCR2:AM106} and [¹⁸F]Z_{DGCR2:AM106} Z-molecule in vitro.

### Methods

Copper Coated Plates ELISA-Based Binding of [⁶⁸Ga]Z_{DGCR2:AM106} and [¹⁸F]Z_{DGCR2:AM106}

Recombinant human DGCR2 His-tag protein (bio-techne, #10161-DG) was purchased and suspended in PBS. The recombinant human DGCR2 His-tag protein was coated at 10 μg/mL onto a Pierce copper coated high capacity plate (Thermofisher) using a 15 min blocking step with PBS containing 1% BSA. [⁶⁸Ga]Z_{DGCR2:AM106} or [¹⁸F]Z_{DGCR2:AM106} was incubated in each well at 5 MBq/mL for 60 min at room temperature (RT) with shaking. A series of 3 washes using PBS containing 0.05% Tween-20 was performed prior to measurement in a Nal well counter (Uppsala Imanet AB, Uppsala, Sweden).

### Results

Copper Coated Plates ELISA-Based Binding of [⁶⁸Ga]Z_{DGCR2:AM106} and [¹⁸F]Z_{DGCR2:AM106}

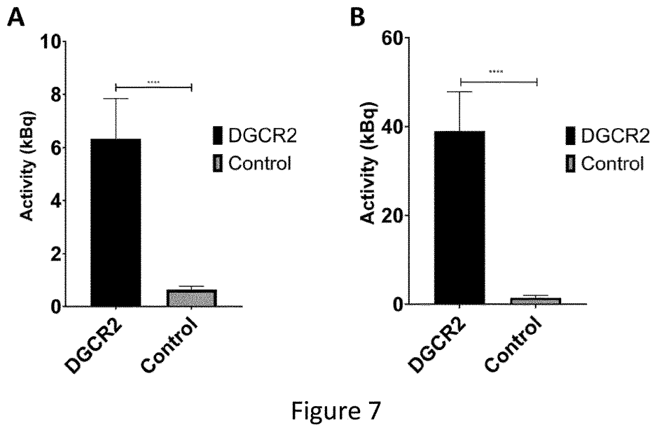

The measured signal of [⁶⁸Ga]Z_{DGCR2:AM106} was significantly higher in wells containing the recombinant human DGCR2 (6.33±1.52 kBq, n=11) compared to the negative control (0.65±0.13 kBq, n=13) (p<0.0001) using the unpaired Student's t-test using p<0.05 as the limit of significance, as shown in FIG. 7.

[¹⁸F]Z_{DGCR2:AM106} showed a significantly higher binding to the Purified recombinant human DGCR2 protein (38.96±8.88 kBq, n=17) compared negative control (1.46±0.57 kBq, n=20) (p*<0.0001) (FIG. 7). Thus, in conclusion, Z_{DGCR2:AM106}, modified and labeled with either ⁶⁸Ga or ¹⁸F retained binding capacity towards human DGCR2 protein.

### Example 3, Characterization of the In Vivo Biodistribution of the Z-Molecule by Conjugation to a Radionuclide

### Aims

This example describes the in vivo biodistribution of the Z-molecules Z_{DGCR2:3D1}, Z_{DGCR2:AM106}, Z_{DGCR2:AM115} and Z_{DGCR2:AM156}. Biodistribution and uptake in tissues was detected by a radiolabel conjugated at the C-terminal of each peptide.

### Methods

### In Vivo SPECT/CT and Organ Biodistribution of the Indium-111 Labeled Z-Molecules in Rats

Sprague Dawley rats were anesthesized via inhalation of 4% vaporized sevoflurane mixed with a 400 mL/min flow of medical air and oxygen. A SPECT/CT was performed following the intravenous injection of 2 MBq (~7 kBq/g) of the Indium-111 labelled Z-molecule (Z_{DGCR2:3D1}, Z_{DGCR2:AM106}, Z_{DGCR2:AM115} or Z_{DGCR2:AM156}) dissolved in PBS through the tail vein of the rat. Manual segmentation and quantification of the kidney were performed on sequential trans-axial projections using the PBAS modeling tool (PMOD technologies LLC). At the end of the scanning period, organs of interest were collected from each rat for gamma radiation counts measurements using a WIZARD Automatic Gamma Counter (PerkinElmer).

In Vivo PET/CT and Organ Biodistribution of [⁶⁸Ga]Z_{DGCR2:AM106} and [¹⁸F]Z_{DGCR2:AM106} in Rats

Sprague Dawley rats were anesthesized via inhalation of 4% vaporized sevoflurane mixed with a 400 mL/min flow of medical air and oxygen. Subsequently, a PET/CT was performed following the intravenous administration of 15 kBq/g (total amount of 5 MBq) of [⁶⁸Ga]Z_{DGCR2:AM106} dissolved in PBS and 10% ethanol through the tail vein of each rat. The PET data were analyzed via manual segmentation using the PBAS image processing tool (PMOD technologies LLC). For the endpoint biodistribution, the rats were first injected with 15 kBq/g [⁶⁸Ga]Z_{DGCR2:AM106} (total amount of 5 MBq) through the lateral tail vein of each rat. Euthanasia of the rats were then conducted 60 min post-injection and organs were collected for measurement in the well counter (Uppsala Imanet AB, Uppsala, Sweden).

Sprague Dawley rats were anesthesized via inhalation of 4% vaporized sevoflurane mixed with a 400 mL/min flow of medical air and oxygen. Subsequently, a PET/MRI following the intravenous administration of 50 kBq/g of [¹⁸F]Z_{DGCR2:AM106} dissolved in PBS and 10% ethanol through the tail vein of each rat. The PET data was analyzed with manual segmentation on sequential transaxial projections using the PBAS modeling tool (PMOD technologies LLC). For the endpoint biodistribution, the rats were first injected with 20 kBq/g of [¹⁸F]Z_{DGCR2:AM106} through the lateral tail vein of each rat. Euthanasia of the rats was then conducted 90 min post-injection and organs were collected for measurement in the well counter (Uppsala Imanet AB, Uppsala, Sweden).

In Vivo Pig PET/CT Imaging of [¹⁸F]Z_{DGCR2:AM106}

Yorkshire x Swedish Landrace x Hampshire pigs were anesthetized via intravenous administration through the ear of a mixture of ketamine, fentanyl and midazolam. The pigs were then connected to an intratracheal ventilator and a central venous catheter for tracer infusion. A PET/CT was performed following the administration of 2 MBq/kg of [¹⁸F]Z_{DGCR2:AM106} dissolved in 2 mL of PBS. The PET data was analyzed with manual segmentation on sequential transaxial projections using the PBAS modeling tool (PMOD technologies LLC).

### Results

### In Vivo SPECT/CT and Organ Biodistribution of the Indium-111 Labeled Z-Molecules in Rats

The SPECT/CT images revealed a strong clearance through the kidneys with marginal liver excretion. ¹¹¹In-DOTA-Z_{DGCR2:AM156} showed a slow renal uptake profile compared to the other Z-molecules while ¹¹¹In-DOTA-Z_{DGCR2:3D1} displayed the highest renal uptake.

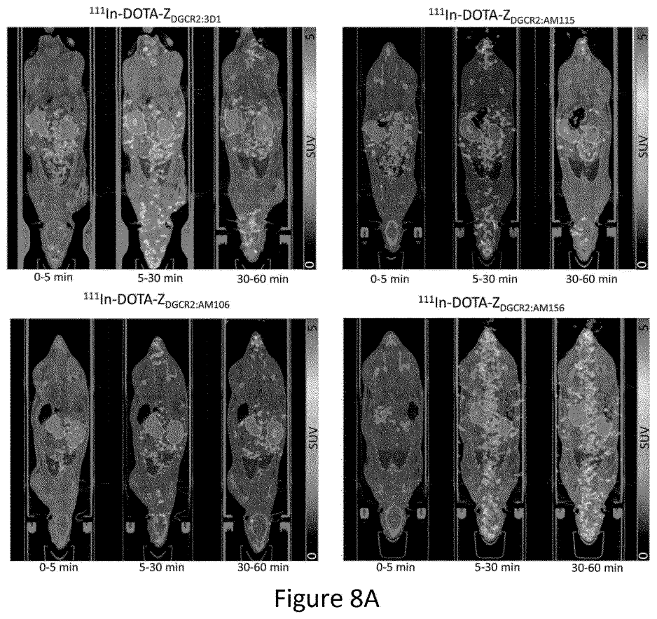

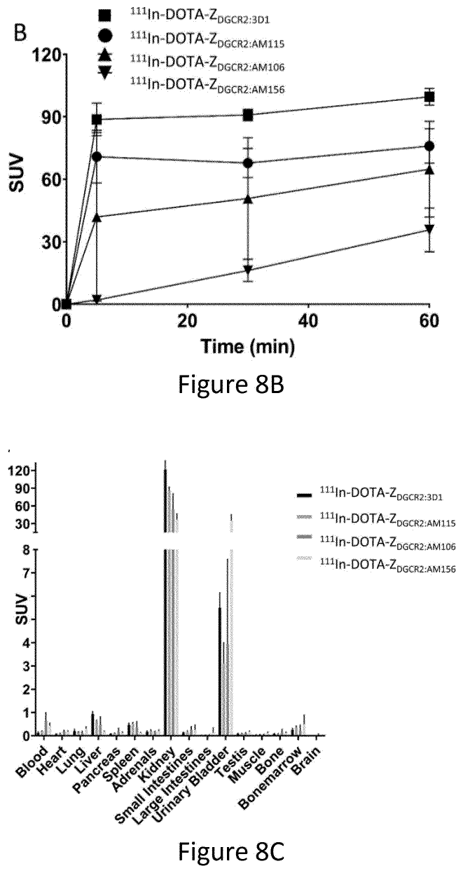

The endpoint biodistribution profile at 60 min post-injection indicated a high radioactivity signal in the kidney and subsequently in the urinary bladder for ¹¹¹In-DOTA-Z_{DGCR2:3D1} (SUV_{kidney}=121±15, n=3), ¹¹¹In-DOTA-Z_{DGCR2:AM106} (SUV_{kidney}=54±26, n=3), ¹¹¹In-DOTA-Z_{DGCR2:AM115} (SUV_{kidney}=85±6, n=3) and ¹¹¹In-DOTA-Z_{DGCR2:AM156} (SUV_{kidney}=37±9, n=3) (FIG. 8). Background signal in most tissues was generally low (SUV<1) for all the candidates, indicating that high contrast imaging could be accomplished given the presence of human DGCR2 expression. Furthermore, the rapid excretion is indicative of suitable dosimetry allowing repeated dosing in humans.

In Vivo PET/CT and Organ Biodistribution of [⁶⁸Ga]Z_{DGCR2:AM106} and [¹⁸F]Z_{DGCR2:AM106} in Rats

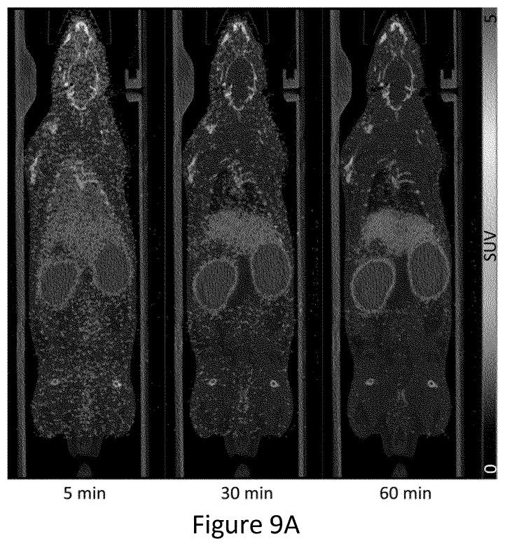

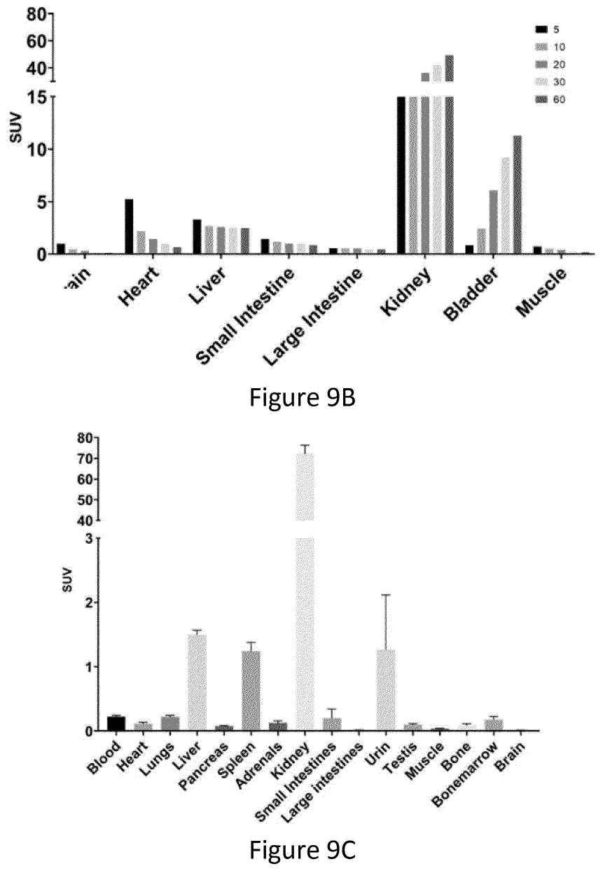

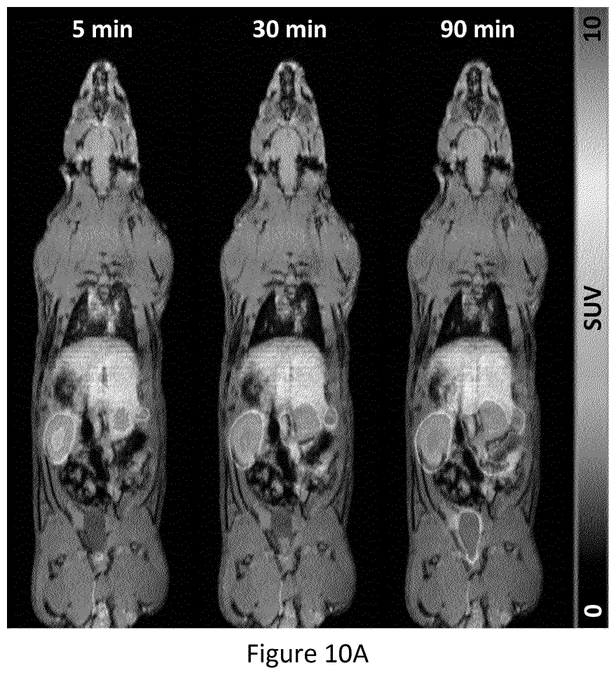

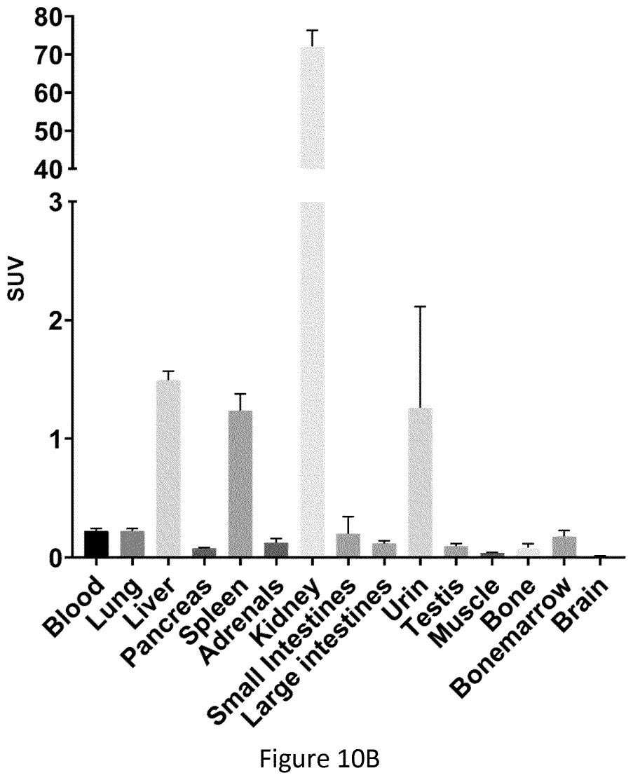

The dynamic biodistribution of [⁶⁸Ga]Z_{DGCR2:AM106} in the rat over the 60 min PET/CT demonstrated mainly a renal clearance. The endpoint biodistribution from the collected organs aligned with the dynamic PET/CT results with [⁶⁸Ga]Z_{DGCR2:AM106} mainly excreted through renal clearance along with a baseline liver and spleen signal (FIG. 9). The dynamic biodistribution of [¹⁸F]Z_{DGCR2:AM106} in the rat over the 90 min PET/MRI demonstrated mainly a renal clearance along with low liver uptake (FIG. 10). The endpoint biodistribution from the collected organs aligned with the dynamic PET/CT results, showing a mainly renal clearance of [¹⁸F]Z_{DGCR2:AM106} along with a baseline liver and spleen signal due to their naturally high vascularization and perfusion. In conclusion, both [⁶⁸Ga]Z_{DGCR2:AM106} and [¹⁸F]Z_{DGCR2:AM106} exhibited rapid clearance and generally low tissue background, demonstrating low off target binding, and that high contrast imaging would be feasible if human DGCR2 was present in tissue.

In Vivo Pig PET/CT Imaging of [¹⁸F]Z_{DGCR2:AM106}

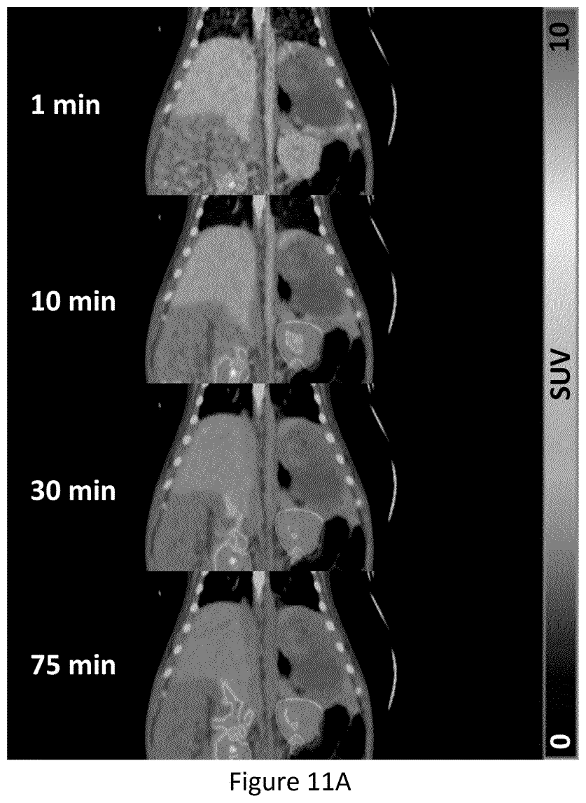

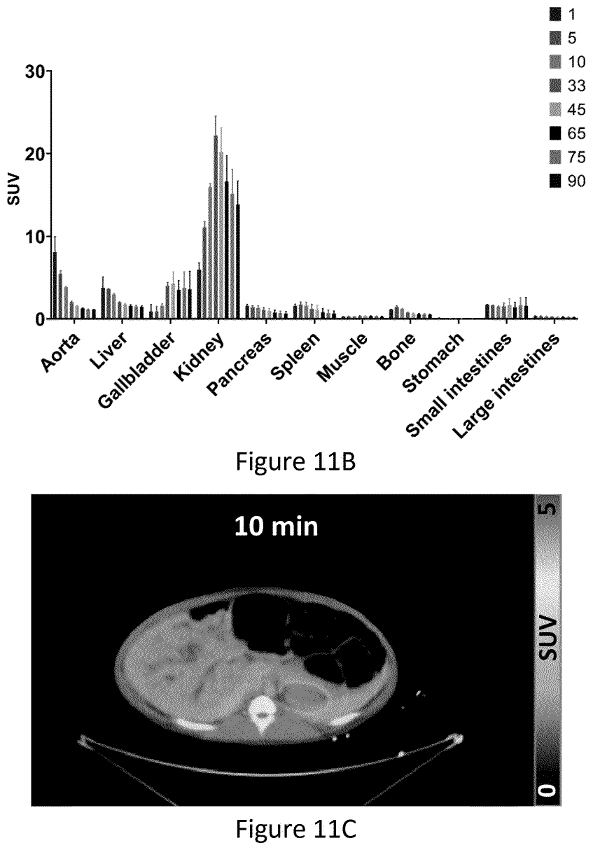

The PET/CT images in the pigs showed rapid distribution of [¹⁸F]Z_{DGCR2:AM106} already 1 min after venous administration, with a brief passage through the lungs. Early uptake could be observed in the liver but was gradually cleared over the duration of the scan. A strong renal clearance could be observed with a peak at 30 min p.i which persisted through the examination along with a marginal uptake in the other abdominal organs. Both the pancreas and the spleen exhibited uptake of [¹⁸F]Z_{DGCR2:AM106} but rapidly decrease over time (SUV_{60 min}<1) (FIG. 11). Background tissue in most tissues was low, showing the potential of high contrast imaging combined with low off target binding.

### Example 4, In Vivo Delivery of an Imaging Reagent to Transplanted Islets by Z_{DGCR2:AM106}

### Aims

This example describes the use of Z_{DGCR2:AM106} Z-molecule for use in vivo to deliver an imaging reagent (¹⁸F) to transplanted islet grafts, monitored by PET.

### Methods

In Vivo PET/MRI and Organ Biodistribution of Mice with Intramuscular Islet Grafts

NMRI nu/nu mice (Taconic M&B, Ejby, Denmark) were transplanted with up to 800 human islets to the neck muscle. Mice were anesthetized with isoflurane before exposition of the muscle. Approximately four weeks after transplantation, for each mouse, a PET/MRI acquisition was performed for up to 60 minutes following the administration of [¹⁸F]Z_{DGCR2:AM106}, 400 kBq/g, through the lateral vein.

### Results

In Vivo PET/MRI and Organ Biodistribution of Mice with Intramuscular Islet Grafts

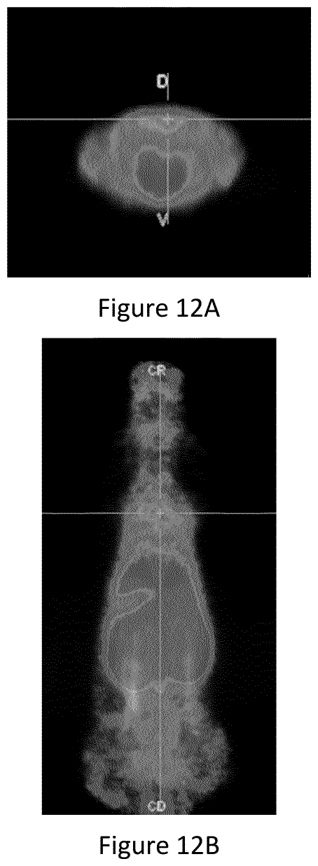

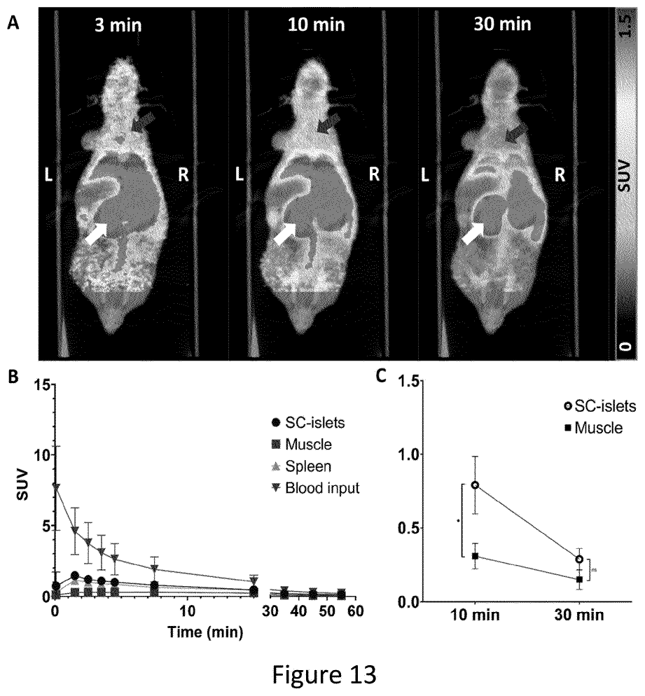

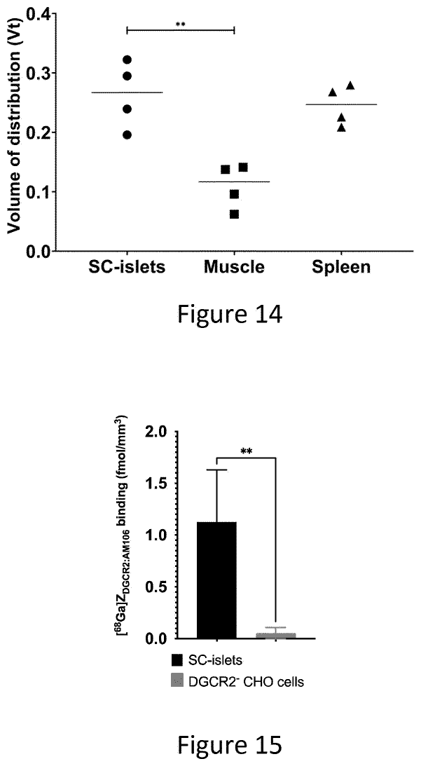

The site of islets grafted in the neck were clearly identifiable and visible after administration of [¹⁸F]Z_{DGCR2:AM106}. Thus, the Z_{DGCR2:AM106} peptide can be used to deliver imaging reagents to human islets in vivo, combined with low off target binding. FIG. 12 shows PET images of [¹⁸F]Z_{AM106} binding in mice with intramuscular islet grafts in the neck. FIG. 13 shows the distribution with time. FIG. 14 shows the volume of distribution of [¹⁸F]Z_{DGCR2:AM106} in SC-islets, muscle and spleen.

### Example 5, In Vivo Delivery of Extracellular Vesicles Conjugated to DGCR2 Binding Polypeptide to Transplanted Islets

### Aims

This example describes the delivery of an EV based drug towards human beta cells in vivo, by conjugating the EV to a DGCR2 targeting polypeptide.

### Methods

Immunodeficient mice are transplanted with up to human islets to the neck muscle or subcutaneous space. EVs are prepared from human Mesenchymal Stromal Cells (MSCs). The DGCR2 binding polypeptide is modified at the N terminus by a lipophilic anchor. After mixing, this functionalization will anchor the DGCR2 targeting moeity into the EV cell membrane. The EVs are also labeled by an intracellular fluorescent marker (CellTracker).

The EV-DGCR2 targeting construct is administered intravenously to the mice, and the accumulation in the human islets monitored postmortem by fluorescent imaging and histology.

### Results

The experiment provides proof of principle (POP) of in vivo beta-cell selective targeting of EVs assisted by the DGCR2 targeting guiding moiety.

### Example 6, In Vitro Autoradiography on Stem Cell Derived Islets (SC-Islets)

### Methods

Binding of the Z-molecules was evaluated by in vitro autoradiography. Briefly, SC-islets were embedded in OCT and frozen to −80° C. SC-islets were cultured as described previously. The frozen cell pellet of non-transfected CHO cells with negligible expression of DGCR2 was used as comparison. Each preparation was processed into 20 μm slices using a cryotome, and placed upon object glasses (Superfrost Plus, positively charged to increase tissue adhesion).

Sections were first pre-incubated in assay buffer (PBS, pH 7.4, 150 mL) for 10 minutes. Next, radiolabeled Z-molecule [68Ga]ZAM106 was added in a concentration of 6.1 nM.

The sections were incubated for 40 minutes at room temperature, and then washed 3×3 minutes in 150 mL of cold PBS (4° C., pH 7.4) to remove excess unbound ligand, and then airdried at 37° C. for 10 minutes. The sections were then exposed to a digital phosphor-imager screen for 1 hour before being developed using a phosphorimager scanner (Typhoon, GE Healthcare). A 10 μl reference of known radioactivity was included to allow for quantification of the results.

### Results

[68Ga]ZAM106 exhibited strong binding to SC-islets, while binding to negative control cells was negligible (FIG. 15). The binding intensity, approximately 1 fmol/mm³, corresponds to 1.4 μmol per islet (assuming an islet equivalent diameter of 150 μm), which equals in the range of 850.000 bound [⁶⁸Ga]Z_{DGCR2:AM106} ligands per islet at this concentration.

### Example 6, Effect of In Vitro ZDGCR2:AM106 Exposure to Human Isolated Islets

### Methods

The potential effect on islet viability and e.g. insulin secretion was evaluated at different doses of ZAM106. Isolated islets from human donors (n=2) were cultured with different doses of ZAM106 for 48 hours (75 islets per conditions; 60 μM [high dose], 60 nM [low dose], 0 nM [control]). The islets were counted and microscoped before and after incubation. After the incubation, the glucose-stimulated insulin secretion (GSIS) was measured by perifusion of 20 islets from each condition, at 37° C. After washing with low glucose for stabilization, islets were stimulated with high glucose from 28-60 minutes. Aliquots from outflow tubing was continuously collected and later analysed for insulin content by ELISA.

### Results

Incubation of islets with ZAM106 at doses up to 60 μM did not have any apparent effect on islet number or viability. Islet perifusion experiments demonstrated a characteristic biphasic pattern of insulin release during stimulation with high glucose. Exposure to ZAM106 did not inhibit or potentiate glucose induced insulin secretion, which may interfere with delivery drugs intending to affect the same or cause unwanted side effects such as hypoglycemia.

1. A binding polypeptide that specifically binds DGCR2, wherein the DGCR2-binding polypeptide comprises a DGCR2 binding motif, DBM, which motif consists of the amino acid sequence selected from:
(SEQ ID NO: 1)
i) EQX₃HARNEILX₁₁LPNLNRMQX₂₀X₂₁AFIX₂₅
wherein, independently of each other,
X₃ is selected from T and Y;
X₁₁ is selected from Q and R;
X₂₀ is selected from K, R and H;
X₂₁ is selected from S, M, L or T;
X₂₅ is selected from R, Q, or K;
and
ii) an amino acid sequence which has at least 88% identity to the sequence defined in i), such as 92% or 96% identity thereto.

2. The binding polypeptide of claim 1, wherein the DGCR2-binding polypeptide binds to DGCR2 such that the K_{D} value of the interaction is at most 1 μM.

3. The binding polypeptide of claim 1, wherein said DGCR2 binding motif forms part of a three-helix bundle protein domain, and wherein in which said DGCR2 binding motif essentially forms part of two alpha helices with an interconnecting loop, within said three-helix bundle protein domain.

4. The binding polypeptide of claim 1, wherein DGCR2-binding polypeptide comprises an amino acid sequence selected from:
(SEQ ID NO: 2)
VDNKFNK-[DBM]-SLIDDPSQSANLLAEAKKLNDAQAPK;
and
(SEQ ID NO: 3)
VDNKFNK-[DBM]-SLIDDPSQSANLLAEAKKLNDAQAPKC,
wherein [DBM] is a DGCR2 binding motif as defined in claim 1.

5. The binding polypeptide of claim 1, wherein the binding polypeptide comprises an amino acid sequence selected from SEQ ID NO: 11-24.

6. A conjugated binding molecule comprising:
i) at least one binding polypeptide as defined in claim 1; and
ii) at least one agent.

7. The conjugated binding molecule of claim 6, wherein the at least one agent comprises a therapeutic agent and/or a detectable agent.

8. The conjugated binding molecule of claim 7, wherein the binding molecule comprise at least one therapeutic agent and at least one detectable agent binding, wherein the therapeutic agent is conjugated to one of the N-terminus or C-terminus of the binding polypeptide, and the detectable agent is conjugated to the other of the N-terminus or C-terminus of the binding polypeptide.

9. The conjugated binding molecule of claim 7, wherein the at least one therapeutic agent is an agent used for treatment of beta-cell related disorders and/or beta-cell dysfunction, such as diabetes.

10. The conjugated binding molecule of claim 7, wherein the at least one therapeutic agent is selected from small molecules, peptides, proteins, biologic drugs, nucleic acids and cells, such as mesenchymal stem cells, extracellular vesicles and regulatory T cells.

11. The conjugated binding molecule of claim 7, wherein the at least one detectable agent is selected from the group consisting of radioisotopes, contrast agents, enzymes, fluorescent molecules, dyes, digoxigenin, and biotin.

12. The conjugated binding molecule of claim 6, wherein the at least one agent is indirectly joined to the binding polypeptide via a linker in the form of a chelator, functional group or prosthetic group, wherein the at least one agent comprises or consists of a radionuclide.

13. The conjugated binding molecule of claim 12, wherein the chelator is selected from the group consisting of derivatives of 1,4,7,10-tetraazacyclododecane-1,4,7,10, tetraacetic acid (DOTA), derivatives of deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA), derivatives of (tBu) 4 (1-(1-carboxy-3-carbotertbutoxypropyl)-4,7,10-(carbotertbut-oxymethyl)-1,4,7,10-tetraazacyclododecane) (DOTAGA), derivatives of 1,4,8,11-tetraazacyclodocedan-1,4,8,11-tetraacetic acid (TETA), derivatives of 1,4,7-triazacyclononane, 1-glutaric acid-4,7-acetic acid (NODAGA), derivatives of 1,4,7-Triazacyclononane-1,4,7-triacetic acid (NOTA), and Restrained Complexing Agent (RESCA).

14. The conjugated binding molecule of claim 11, wherein the radioisotope is selected from the group consisting of ⁶⁸Ga, ¹¹C, ¹⁸F, ⁶⁴Cu, ⁸⁹Zr, ⁴⁴Sc, ⁵²Mn, ⁵⁵Co, ⁷²As, ¹¹¹In, ^{99m}Tc, ⁶⁷Ga, ⁷²As, ¹²³I, ¹²⁴I, ¹²⁵I, ¹²⁴I, ⁴⁷Sc, ¹⁸⁸Re, ²⁰¹Tl, ¹⁷⁷Lu, ⁹⁰Y, Gd, ²¹³Bi, ²²⁵Ac, ²¹²Pb and ²¹¹At.

15. The conjugated binding molecule of claim 11, wherein the detectable agent is detectable by an imaging technique such as PET, MRI, SPECT, Computed Tomography (CT), optical or ultrasound imaging.

16-21. (canceled)

22. The conjugated binding molecule of claim 6 for use in Position Emission Tomography (PET) imaging, wherein the conjugated binding molecule comprises at least one agent comprising a detectable agent.

23. (canceled)


SEQUENCE LISTING (24 sequences)

SEQ ID NO: 1 - protein, 25 aa, synthetic construct
  VARIANT 3: note = T or Y
  VARIANT 11: note = Q or R
  VARIANT 20: note = K, R or H
  VARIANT 21: note = S, M, L or T
  VARIANT 25: note = R, Q, or K
EQXHARNEILXLPNLNRMQXXAFIX

SEQ ID NO: 2 - protein, 58 aa, synthetic construct
  VARIANT 10: note = T or Y
  VARIANT 18: note = Q or R
  VARIANT 27: note = K, R or H
  VARIANT 28: note = S, M, L or T
  VARIANT 32: note = R, Q, or K
VDNKFNKEQXHARNEILXLPNLNRMQXXAFIXSLIDDPSQSANLLAEAKKLNDAQAPK

SEQ ID NO: 3 - protein, 59 aa, synthetic construct
  VARIANT 10: note = T or Y
  VARIANT 18: note = Q or R
  VARIANT 27: note = K, R or H
  VARIANT 28: note = S, M, L or T
  VARIANT 32: note = R, Q, or K
VDNKFNKEQXHARNEILXLPNLNRMQXXAFIXSLIDDPSQSANLLAEAKKLNDAQAPKC

SEQ ID NO: 4 - protein, 25 aa, synthetic construct
EQTHARNEILQLPNLNRMQKSAFIR

SEQ ID NO: 5 - protein, 25 aa, synthetic construct
EQYHARNEILQLPNLNRMQKMAFIQ

SEQ ID NO: 6 - protein, 25 aa, synthetic construct
EQTHARNEILQLPNLNRMQKSAFIK

SEQ ID NO: 7 - protein, 25 aa, synthetic construct
EQTHARNEILRLPNLNRMQHLAFIQ

SEQ ID NO: 8 - protein, 25 aa, synthetic construct
EQTHARNEILQLPNLNRMQRSAFIQ

SEQ ID NO: 9 - protein, 25 aa, synthetic construct
EQYHARNEILQLPNLNRMQRSAFIQ

SEQ ID NO: 10 - protein, 25 aa, synthetic construct
EQTHARNEILQLPNLNRMQRTAFIK

SEQ ID NO: 11 - protein, 58 aa, synthetic construct
VDNKFNKEQTHARNEILQLPNLNRMQKSAFIRSLIDDPSQSANLLAEAKKLNDAQAPK

SEQ ID NO: 12 - protein, 58 aa, synthetic construct
VDNKFNKEQYHARNEILQLPNLNRMQKMAFIQSLIDDPSQSANLLAEAKKLNDAQAPK

SEQ ID NO: 13 - protein, 58 aa, synthetic construct
VDNKFNKEQTHARNEILQLPNLNRMQKSAFIKSLIDDPSQSANLLAEAKKLNDAQAPK

SEQ ID NO: 14 - protein, 58 aa, synthetic construct
VDNKFNKEQTHARNEILRLPNLNRMQHLAFIQSLIDDPSQSANLLAEAKKLNDAQAPK

SEQ ID NO: 15 - protein, 58 aa, synthetic construct
VDNKFNKEQTHARNEILQLPNLNRMQRSAFIQSLIDDPSQSANLLAEAKKLNDAQAPK

SEQ ID NO: 16 - protein, 58 aa, synthetic construct
VDNKFNKEQYHARNEILQLPNLNRMQRSAFIQSLIDDPSQSANLLAEAKKLNDAQAPK

SEQ ID NO: 17 - protein, 58 aa, synthetic construct
VDNKFNKEQTHARNEILQLPNLNRMQRTAFIKSLIDDPSQSANLLAEAKKLNDAQAPK

SEQ ID NO: 18 - protein, 59 aa, synthetic construct
VDNKFNKEQTHARNEILQLPNLNRMQKSAFIRSLIDDPSQSANLLAEAKKLNDAQAPKC

SEQ ID NO: 19 - protein, 59 aa, synthetic construct
VDNKFNKEQYHARNEILQLPNLNRMQKMAFIQSLIDDPSQSANLLAEAKKLNDAQAPKC

SEQ ID NO: 20 - protein, 59 aa, synthetic construct
VDNKFNKEQTHARNEILQLPNLNRMQKSAFIKSLIDDPSQSANLLAEAKKLNDAQAPKC

SEQ ID NO: 21 - protein, 59 aa, synthetic construct
VDNKFNKEQTHARNEILRLPNLNRMQHLAFIQSLIDDPSQSANLLAEAKKLNDAQAPKC

SEQ ID NO: 22 - protein, 59 aa, synthetic construct
VDNKFNKEQTHARNEILQLPNLNRMQRSAFIQSLIDDPSQSANLLAEAKKLNDAQAPKC

SEQ ID NO: 23 - protein, 59 aa, synthetic construct
VDNKFNKEQYHARNEILQLPNLNRMQRSAFIQSLIDDPSQSANLLAEAKKLNDAQAPKC

SEQ ID NO: 24 - protein, 59 aa, synthetic construct
VDNKFNKEQTHARNEILQLPNLNRMQRTAFIKSLIDDPSQSANLLAEAKKLNDAQAPKC